(19) United States
(12) Patent Application Publication
(10) Pub. No.: US 2022/0411449 A1
(43) Pub. Date: Dec. 29, 2022

(54) Method for Preparing Heteroleptic Triarylbismuthanes and Compounds Produced by the Same

(71) Applicant: UNIVERSITY OF HAWAII, Honolulu, HI (US)

(72) Inventors: Jakub HYVL, Honolulu, HI (US); Axel LEHRER, Honolulu, HI (US)

(21) Appl. No.: 17/774,723

(22) PCT Filed: Nov. 13, 2020
(86) PCT No.: PCT/US2020/060339

§ 371 Date: May 5, 2022

Related U.S. Application Data
(60) Provisional application No. 62/934,943, filed on Nov. 13, 2019.

(51) Int. Cl. C07F 9/94; A61P 31/12; A61P 31/04

(52) U.S. Cl. CPC C07F 9/94; A61P 31/12; A61P 31/04

(57) ABSTRACT

A method for controlling dismutation in the synthesis of a heteroleptic triarylbismuthane is provided as are compounds produced by such a method and use of the same to inhibit the replication of microorganisms.

### INTRODUCTION

This application claims benefit of priority to U.S. Provisional Patent Application Ser. No. 62/934,943, filed Nov. 13, 2019, the content of which is incorporated herein by reference in its entirety.

### BACKGROUND

The growing interest in organobismuth chemistry is driven by applications in synthetic chemistry, catalysis, medicinal chemistry, and material science without the toxicity and environmental risks typically associated with its heavy-atom congeners. Despite its potential, organobismuth chemistry is far less developed in comparison with other main-group organometallic chemistry due to two obstacles. First, the C—Bi bond is weak (bond dissociation energy (BDE)=46 kcal/mol), making it prone to homolytic cleavage. Second, dismutation, a substituent scrambling process occurs wherein tri(p-tolyl)bismuthane can exchange substituents with n-butyllithium (Eq 1; Challenger (1914) J. Chem. Soc. Trans. 105:2210-8; Gilman, et al. (1939) J. Am. Chem. Soc. 61(5):1170-2). This complicates a selective synthesis of unsymmetrical organobismuthanes of general formula Ar¹₂Ar²Bi (Ar¹≠Ar²) (Gilman & Yablunky (1941) J. Am. Chem. Soc. 63(1):207-211).

(p-Tol)₃Bi+3n-BuLi [symbol] 3p-TolLi+(n-Bu)₃Bi (1)

The same exchange process has been observed for all triaryl pnictogens, except nitrogen, and an ate-complex has been proposed as the mechanism of this process (Wittig & Naercker (1967) J. Organomet. Chem. 8(3):491-4). This ate-complex was suggested to be an intermediate responsible for the formation of side product, PhBi(n-Bu)₂, in addition to the main product, Ph₂Bi(n-Bu), generated from reaction of Ph₂BiCl and n-BuLi (Eq 2; Kauffmann, et al. (1985) Chem. Ber. 118(3):1031-8).

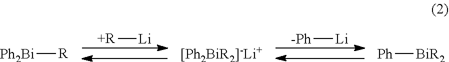

Furthermore, it has been observed that the unsymmetrical triarylbismuthanes Ar¹₂Ar²Bi, where Ar¹≠Ar², are also prone to dismutation (Eq 3; Barton, et al. (1986) Tetrahedron 42(12):3111-3122). On the basis of this report, unsymmetrical triarylbismuthanes were viewed as an unstable and elusive species.

Ar¹₂Ar²Bi [symbol] Ar¹Ar²₂Bi [symbol] Ar¹₃Bi⁺Ar²₃Bi (3)

Besides the ate-complex-promoted mechanism of dismutation, other mechanistic modes have been proposed. For the monoaryldialkylbismuthanes, a light-induced dismutation was observed (Wieber & Sauer (1985) Z. Naturforsch B: J. Chem. Sic. 40(11):1476-80), suggestive of a radical mechanism, and in the presence of bismuth trihalides an electrophilic mode likely operates (Barton, et al. (1986) Tetrahedron 42(12):3111-3122). Moreover, other factors are also involved; for example, steric congestion around the bismuth center can suppress the dismutation process completely (Matano, et al. (1992) Bull. Chem. Soc. Jpn. 65(12):3504-6).

### SUMMARY OF THE INVENTION

This invention is a method of controlling dismutation in the synthesis of a heteroleptic triarylbismuthane which includes adding to a nucleophile a substoichiometric amount of a diarylbismuth or arylbismuth precursor relative to said nucleophile so that dismutation of the heteroleptic triarylbismuthane is controlled. In some embodiments, the diarylbismuth or arylbismuth precursor has the structure of Formula I

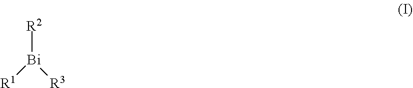

wherein R¹ is a substituted or unsubstituted aryl; R² is a leaving group, in particular a tosyl group; and R³ is the same as R¹ or R²; the nucleophile is an organometal such as an organozinc, organomagnesium or organocuprate; and the heteroleptic triarylbismuthane has the structure of Formula II

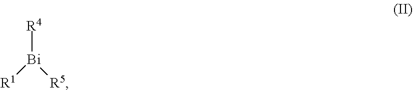

wherein R¹ and R⁴ are independently a substituted or unsubstituted aryl; R⁵ is the same as R¹ or R⁴; and R¹ and R⁴ are different.

A compound having the structure of Formula II and pharmaceutical composition containing the same are also provided, wherein Formula IT is

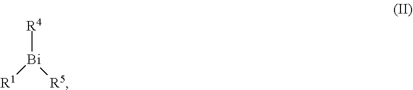

and wherein R¹ and R⁴ are independently a substituted or unsubstituted aryl or substituted or unsubstituted arylsulfonate; and R⁵ is the same as R¹ or R⁴, wherein at least one of R¹ or R⁴ is

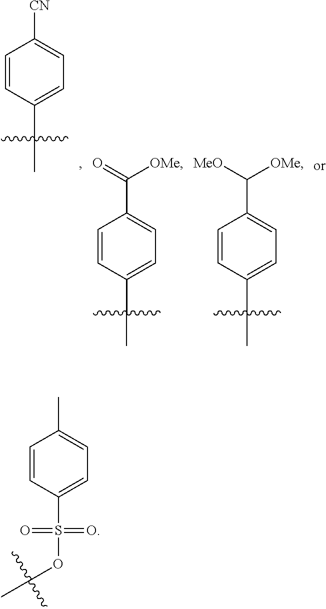 [SMILES: *c1ccc(C#N)cc1 ; *c1ccc(C(=O)OC)cc1 ; *c1ccc(C(OC)OC)cc1 ; *OS(=O)(=O)c1ccc(C)cc1]

The invention further provides a method for inhibiting the replication of a microorganism by contacting a microorganism with a compound of the invention, wherein in certain embodiments the microorganism is a virus or bacterium.

### DETAILED DESCRIPTION OF THE INVENTION

Heteroleptic triarylbismuthanes, Ar¹₂Ar²Bi, where Ar¹≠Ar², are an unexplored class of metallodrugs with various biological activities. In addition, these compounds exhibit good lipophilicity and good structural variability. A simple method for synthesizing heteroleptic triarylbismutanes with a variety of functional groups has now been developed, which consistently gives yields in the range of 80-99%. When the Ar¹₂Ar²Bi product is formed in a high yield, dismutated side products, e.g. Ar¹Ar²₂Bi, are not produced and the Ar¹₂Ar²Bi product can be easily isolated. To avoid dismutation, it has now been shown that addition of the electrophile to the nucleophile and use of substoichiometric amounts of diarylbismuth or arylbismuth precursors (e.g., arylbismuth Cl, Br, sulfonate) relative to the nucleophile are required. Further, the use of a diarylbismuth sulfonate or arylbismuth disulfonate as the precursor in the synthesis of unsymmetrical triaryl bismuthanes improves yields. Using the instant method, a group of novel heteroleptic triarylbismuthanes were produced and shown to exhibit antimicrobial activity. Accordingly, the present invention provides a method of controlling dismutation in the synthesis of a heteroleptic triarylbismuthane, as well as novel compounds and methods of using the same to inhibit the replication of microorganisms.

As is conventional in the art, a heteroleptic compound is an organometallic compound having two or more different types of ligand. In the present invention, the organometallic compound is a triarylbismuthane. In certain aspects, the heteroleptic triarylbismuthane compound of the invention, also referred to herein as an unsymmetrical bismuthane, has the general structure of Ar¹₂Ar²Bi (Ar¹≠Ar²) or Formula II

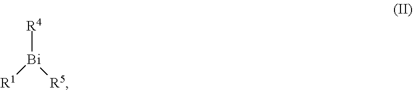

wherein R¹ and R⁴ are independently a substituted or unsubstituted aryl; R⁵ is the same as R¹ or R⁴; and R¹ and R⁴ are different.

For the purposes of the present method, “dismutation,” also commonly referred to as “disproportionation” refers to a chemical reaction where a molecule is transformed into two or more dissimilar products. By way of illustration, dismutated contaminants in the synthesis of the heteroleptic triarylbismuthane Ar¹₂Ar²Bi include, Ar²₂Ar¹Bi, Ar¹₃Bi, and Ar²₃Bi. In accordance with the present method, dismutation in the synthesis of a heteroleptic triarylbismuthane is controlled, inhibited or reduced by (i) adding the electrophile (i.e., a diarylbismuth or arylbismuth precursor) to a nucleophile (as opposed to adding the nucleophile to the electrophile); and (ii) using a substoichiometric amount of the diarylbismuth or arylbismuth precursor relative to the nucleophile.

Ideally, the electrophile used in the present method is a diarylbismuth or arylbismuth precursor. In certain aspects, the diarylbismuth or arylbismuth precursor has the structure of Formula I

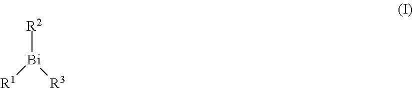

wherein R¹ is a substituted or unsubstituted aryl; R² is a leaving group; and R³ is the same as R¹ or R². In some aspects of the present method, a diarylbismuth precursor is used, i.e., R¹ and R³ are each a substituted or unsubstituted aryl and R² is a leaving group. In other aspects of the present method, an arylbismuth precursor is used, i.e., R¹ is a substituted or unsubstituted aryl and R² and R³ are each a leaving group. In certain aspects, R¹ is a substituted or unsubstituted aryl; R² is a tosyl group; and R³ is the same as R¹ or R². In some aspects, R¹ and R³ are each a substituted or unsubstituted aryl and R² is a tosyl group. In other aspects, R¹ is a substituted or unsubstituted aryl and R² and R³ are each a tosyl group.

“Aryl” refers to a radical of a monocyclic or polycyclic (e.g., bicyclic or tricyclic) aromatic ring system having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Examples of such fused rings include, e.g., indanyl (including, for example, indan-5-yl, or indan-2-yl, and the like) or tetrahydronaphthyl (including, for example, tetrahydronaphth-1-yl, tetrahydronaphth-2-yl, and the like), and the like. In certain embodiments, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl, wherein said substituents may be the same or different.

Substituents which can be used for the substituted aryl group are independently disclosed herein and can be used without limitation to further describe the substituted aryl group. In some embodiments, the substituted aryl group is a substituted phenyl group which can be a 2-substituted phenyl group, a 3-substituted phenyl group, a 4-substituted phenyl group, a 2,4-disubstituted phenyl group, a 2,6-disubstituted phenyl group, 3,5-disubstituted phenyl group, or a 2,4,6-trisubstituted phenyl group.

Examples of substituents independently include (1) halogen atoms (e.g., fluorine atom, chlorine atom, bromine atom, iodine atom, etc.), (2) nitro, (3) cyano, (4) hydroxy or oxo, (5) C₁₋₆ alkoxy optionally having 1 to 3 halogen atoms (e.g., fluorine, chlorine, bromine, iodine) (e.g., methoxy, ethoxy, propoxy, isopropoxy, butoxy, isobutoxy, sec-butoxy, pentyloxy, hexyloxy, fluoromethoxy, etc.), (6) C₆₋₁₄ aryloxy (e.g., phenyloxy, naphthyloxy, etc.), (7) C₇₋₁₆ aralkyloxy (e.g., benzyloxy, phenethyloxy, diphenylmethyloxy, 1-naphthylmethyloxy, 2-naphthylmethyloxy, 2,2-diphenylethyloxy, 3-phenylpropyloxy, 4-phenylbutyloxy, 5-phenylpentyloxy, etc.), (8) mercapto, (9) C₁₋₆ alkylthio optionally having 1 to 3 halogen atoms (e.g., fluorine, chlorine, bromine, iodine) (e.g., methylthio, difluoromethylthio, trifluoromethylthio, ethylthio, propylthio, isopropylthio, butylthio, 4,4,4-trifluorobutylthio, pentylthio, hexylthio, etc.), (10) C₆₋₁₄ arylthio (e.g., phenylthio, naphthylthio, etc.), (11) C₇₋₁₆ aralkylthio (e.g., benzylthio, phenethylthio, diphenylmethylthio, 1-naphthylmethylthio, 2-naphthylmethylthio, 2,2-diphenylethylthio, 3-phenylpropylthio, 4-phenylbutylthio, 5-phenylpentylthio, etc.) (12) amino, (13) mono-C₁₋₆ alkylamino (e.g., methylamino, ethylamino etc.), (14) mono-C₆₋₁₄ arylamino (e.g., phenylamino, 1-naphthylamino, 2-naphthylamino, etc.), (15) mono-C-₇₋₁₆ aralkylamino (e.g., benzylamino, etc.), (16) di-C₁₋₆ alkylamino (e.g., dimethylamino, diethylamino, etc.), (17) di-C₆₋₁₄ arylamino (e.g., diphenylamino, etc.), (18) di-C₇₋₁₆ aralkylamino (e.g., dibenzylamino, etc.), (19) formyl, (20) C₁₋₆ alkyl-carbonyl (e.g., acetyl, propionyl, etc.), (21) C₆₋₁₄ aryl-carbonyl (e.g., benzoyl, 1-naphthoyl, 2-naphthoyl, etc.), (22) carboxyl, (23) C₁₋₆ alkoxy-carbonyl (e.g., methoxycarbonyl, ethoxycarbonyl, propoxycarbonyl, tert-butoxycarbonyl, etc.), (24). C₆₋₁₄ aryloxy-carbonyl (e.g., phenoxycarbonyl, etc.), (25) carbamoyl, (26) thiocarbamoyl, (27) mono-C₁₆ alkyl-carbamoyl (e.g., methylcarbamoyl, ethylcarbamoyl, etc.), (28) di-C₁₋₆ alkyl-carbamoyl (e.g., dimethylcarbamoyl, diethylcarbamoyl, ethylmethylcarbamoyl, etc.), (29) C₆₋₁₄ aryl-carbamoyl (e.g., phenylcarbamoyl, 1-naphthylcarbamoyl, 2-naphthylcarbamoyl, etc.), (30) C₁₋₆ alkylsulfonyl (e.g., methylsulfonyl, ethylsulfonyl, etc.), (31) 06-14 arylsulfonyl (e.g., phenylsulfonyl, 1-naphthylsulfonyl, 2-naphthylsulfonyl, etc.), (32) 01-6 alkylsulfinyl (e.g., methylsulfinyl, ethylsulfinyl, etc.), (33) C₆₋₁₄ arylsulfinyl (e.g., phenylsulfinyl, 1-naphthylsulfinyl, 2-naphthylsulfinyl, etc.), (34) formylamino, (35) C₁₋₆ alkyl-carbonylamino (e.g., acetylamino, etc.), (36) C₆₋₁₄ aryl-carbonylamino (e.g., benzoylamino, naphthoylamino, etc.), (37) C₁₋₆ alkoxy-carbonylamino (e.g., methoxycarbonylamino, ethoxycarbonylamino, propoxycarbonylamino, butoxycarbonylamino, etc.), (38) C₁₋₆ alkylsulfonylamino (e.g., methylsulfonylamino, ethylsulfonylamino, etc.), (39) C₆₋₁₄ arylsulfonylamino (e.g., phenylsulfonylamino, 2-naphthylsulfonylamino, 1-naphthylsulfonylamino, etc.), (40) C₁₋₆ alkyl-carbonyloxy (e.g., acetoxy, propionyloxy, etc.), (41) C₆₋₁₄ aryl-carbonyloxy (e.g., benzoyloxy, naphthylcarbonyloxy, etc.), (42) C₁₋₆ alkoxy-carbonyloxy (e.g., methoxycarbonyloxy, ethoxycarbonyloxy, propoxycarbonyloxy, butoxycarbonyloxy, etc.), (43) mono-C₁₋₆ alkyl-carbamoyloxy (e.g., methylcarbamoyloxy, ethylcarbamoyloxy, etc.), (44) di-C₁₋₆ alkyl-carbamoyloxy (e.g., dimethylcarbamoyloxy, diethylcarbamoyloxy, etc.), (45) 06-14 aryl-carbamoyloxy (e.g., phenylcarbamoyloxy, naphthylcarbamoyloxy, etc.), (46) a 5- to 7-membered saturated cyclic amino optionally containing, besides one nitrogen atom and carbon atom, 1 or 2 kinds of 1 to 4 heteroatoms selected from a nitrogen atom, a sulfur atom and an oxygen atom (e.g., pyrrolidin-1-yl, piperidino, piperazin-1-yl, morpholino, thiomorpholino, hexahydroazepin-1-yl, etc.), (47) a 5- to 10-membered aromatic heterocyclic group containing, besides carbon atom, 1 or 2 kinds of 1 to 4 hetero atoms selected from a nitrogen atom, a sulfur atom and an oxygen atom (e.g., 2-thienyl, 3-thienyl, 2-pyridyl, 3-pyridyl, 4-pyridyl, 2-guinolyl, 3-quinolyl, 4-quinolyl, 5-quinolyl, 8-quinolyl, 1-isoquinolyl, 3-isoquinolyl, 4-isoquinolyl, 5-isoguinolyl, 1-indolyl, 2-indolyl, 3-indolyl, 2-benzothiazolyl, 2-benzo[b]thienyl, 3-benzo[b]thienyl, 2-benzo[b]furanyl, 3-benzo[b]furanyl, etc.), (48) C₁₋₃ alkylenedioxy (e.g., methylenedioxy, ethylenedioxy, etc.), (49) C₃₋₇ cycloalkyl (e.g., cyclopropyl, cyclobutyl, cyclopentyl, cyclohexyl, cycloheptyl, etc.) and combinations thereof. In certain embodiments, the number of the substituents is 1 to 3, e.g., 1, 2 or 3. In particular embodiments, aryl groups are substituted at one or more of the para, meta and/or ortho positions. In particular embodiments, substituents of the aryl group are selected from the group of —CN, —CH₃, —C(=O)OCH₃, and —C(OCH₃)₂.

As used herein, the term “leaving group” is given its ordinary meaning in the art of synthetic organic chemistry and refers to an atom or a group capable of being displaced by a nucleophile. Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and halofoimates. In some cases, the leaving group is a sulfonic acid ester, such as toluenesulfonate (toluenesulfonate or tosyl group, -OTs), methanesulfonate (mesylate, -OMs), p-bromobenzenesulfonyloxy (brosylate, OBs), or trifluoromethanesulfonate (tiflate, -OTf). In certain aspects, the leaving group is a tosylate or tosyl group.

The term “nucleophile” as used herein is a compound or moiety that is reactive towards an electrophile so as to form a covalent bond between the nucleophile and electrophile. Examples of suitable nucleophiles of use in this instant method include, but are not limited to, organometallic compounds (e.g., organomagnesium, organocuprates, organomagnesium, organozinc), nitrogen compounds (amines, diamines, amino acids) or alcohols and their alcohols derivatives. Ideally, in some aspects the nucleophile of the instant method is an organometal. Non-limiting examples of suitable organometals include organomagnesium compounds, organolithium compounds, organotin compounds, organocuprates compounds, organozinc, and organopalladium compounds, metal carbonyls, metallocenes, carbine complexes, and organometalloids (e.g., organoboranes and organosilanes). In some embodiments, the organometal can be selected from the group of R⁶—MgR⁷, R⁶—ZnR⁷, R⁶—Li, (R⁶)ₚ—B(R⁷)₃₋ₚ,(R⁶)_{q}—Sn(R⁷)_{4-q}, R⁶—CuLi, R⁶—CuMgR⁷, R⁶—Cu(CN)MgR⁷, or R⁶—Cu(CN)Li; wherein R⁶ is a substituted or unsubstituted aryl as described elsewhere herein; R⁷ is halogen (—Cl, —Br, —Fl and —I), or substituted or unsubstituted variants of the following: alkyl, alkenyl, cycloalkyl, aryl, arylalkyl, hydroxy (—OH), and alkoxy, wherein if more than one R⁷ is present, the R⁷ groups can optionally be bonded together to form an optionally substituted cycloalkyl (e.g., 9-BBN), optionally substituted cycloalkenyl, optionally substituted heteroalkyl or optionally substituted heteroalkenyl ring; p can be an integer from 1 to 3; and q can be an integer from 1 to 4. In some embodiments, the organometal is R⁶—MgR⁷, R⁶—ZnR⁷, R⁶—CuLi, R⁶—CuMgR⁷, R⁶—Cu (CN) MgR⁷.

The term “alkyl” refers to a saturated hydrocarbon radical which may be straight chain (for example, methyl, ethyl, propyl or butyl) or branched-chain (for example, isopropyl, t-amyl, or 2,5-dimethylhexyl) or cyclic (i.e., cycloalkyl, for example, cyclobutyl, cyclopropyl or cyclopentyl). This definition applies both when the term is used alone and when it is used as part of a compound term, such as “aralkyl” and similar terms. Preferred alkyl groups are those containing from 1 to 8 carbon atoms, and are referred to as a “(Ci-Ce)alkyl” group. Other preferred alkyl groups include those containing from 1 to 20 carbon atoms. Accordingly, in some embodiments, the alkyl group can be a methyl group, an ethyl group, a propyl group, a butyl group, a pentyl group, a hexyl group, a heptyl group, an octyl group, a nonyl group, a decyl group, a undecyl group, a dodecyl group, a tridecyl group, a tetradecyl group, a pentadecyl group, a hexadecyl group, a heptadecyl group, an octadecyl group, a n-propyl group, an iso-propyl group, a n-butyl group, an iso-butyl group, a sec-butyl group, a tert-butyl group, a n-pentyl group, an iso-pentyl group, a sec-pentyl group, or a neopentyl group.

The term “alkenyl,” as used herein, refers to a moiety which contains one or more sites of unsaturation. The term “alkenyl” as used herein may also refer to a moiety which contains at least one carbon-carbon double bond and includes straight-chain, branched chain and cyclic groups. Alkenyl groups may be optionally substituted. The terms “vinyl” and “olefinic” are sometimes used interchangeably with the term alkenyl. Illustrative alkenyl groups can include, but are not limited to, an ethenyl, group, a propenyl group, a butenyl group, a pentenyl group, a hexenyl group, a heptenyl group, an octenyl group, a nonenyl group, a decenyl group, a undecenyl group, a dodecenyl group, a tridecenyl group, a tetradecenyl group, a pentadecenyl group, a hexadecenyl group, a heptadecenyl group, or an octadecenyl group.

The term “alkoxy” refers to an alkyl group as described above which also bears an oxygen substituent which is capable of covalent attachment to another hydrocarbon radical (such as, for example, methoxy, ethoxy and t-butoxy).

A substoichiometric amount or quantity of a diarylbismuth or arylbismuth precursor relative to the nucleophile refers to the use of less than the stoichiometric amount of diarylbismuth or arylbismuth precursor relative to the nucleophile. Typically, a substoichiometric amount of diarylbismuth or arylbismuth precursor is 0.1 to 0.99 equivalents with respect to the amount of nucleophile, or any amount or range therebetween. Ideally, a substoichiometric amount of diarylbismuth or arylbismuth precursor is in the range of 0.4 to 0.85 equivalents with respect to the amount of nucleophile.

By adding a substoichiometric amount of a diarylbismuth or arylbismuth to a nucleophile, a heteroleptic triarylbismuthane is produced. Preferably, the resulting product is devoid of or contains 30% dismutated contaminants. In certain aspects, the product of the instant method contains less than 30%, 25%, 20%, 15%, 10%, 9%, 8%, 7%, 6%, 5%, 4%, 3%, 2% or 1% dismutated contaminants.

Another aspect of this invention are compounds having the structure of Formula II

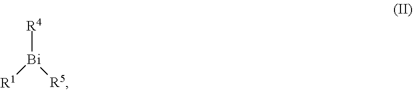

wherein R¹ and R⁴ are independently a substituted or unsubstituted aryl (as described elsewhere herein) or substituted or unsubstituted arylsulfonate; and R⁵ is the same as R¹ or R⁴, wherein at least one of R¹ or R⁴ is

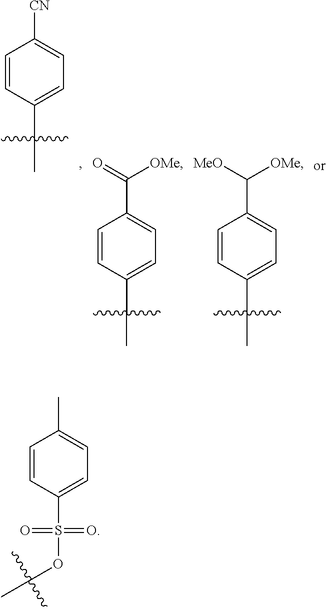 [SMILES: *c1ccc(C#N)cc1 ; *c1ccc(C(=O)OC)cc1 ; *c1ccc(C(OC)OC)cc1 ; *OS(=O)(=O)c1ccc(C)cc1]

An arylsulfonate has the structure Ar—SO₃ and may be unsubstituted or substituted at one or more positions with one or more of the substituents described herein.

Preferred compounds of the present invention have the following structures:

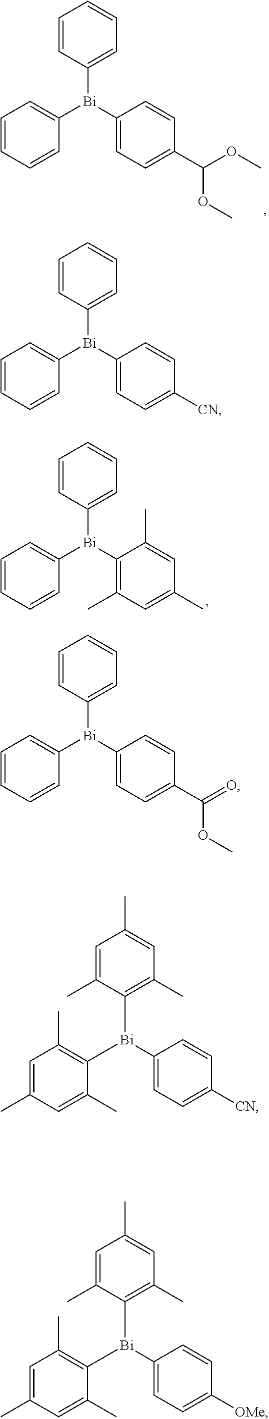

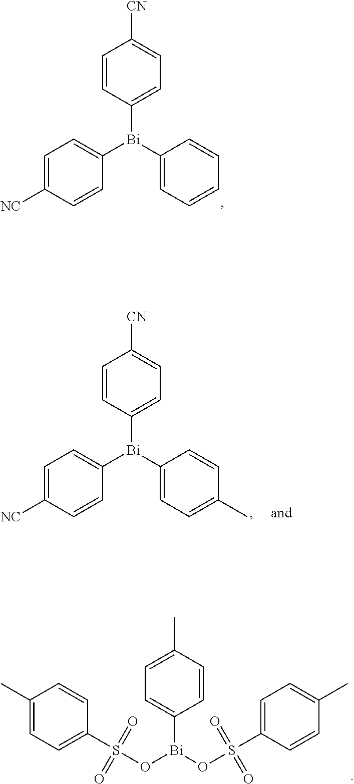

The compounds of this invention may be provided as isolated compounds (e.g., greater than 80%, 85%, 90%, 95%, 96%, 97%, 98% or 99% purity), as a pharmaceutically acceptable salt or in a pharmaceutical composition where said compounds are in admixture with a pharmaceutically acceptable carrier. The term “pharmaceutically acceptable salt” refers to a salt which, upon administration to a recipient is capable of providing (directly or indirectly) a compound as described herein. The preparation of salts can be carried out by methods known in the art. Preferably, “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the federal or a state government or listed in the U.S. Pharmacopeia or other generally recognized pharmacopeia for use in animals, and more particularly in humans.

For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium, ammonium, magnesium, aluminum and lithium salts, and organic alkali salts such as, for example, ethylenediamine, ethanolamine, N,N-dialkylenethanolamine, triethanolamine, glucamine and basic amino acids salts.

Pharmaceutical compositions including a compound of Formula II, or pharmaceutically acceptable salt thereof, and at least one pharmaceutically acceptable carrier, adjuvant, and/or vehicle, are also provided. “Carriers” are substances which improve the delivery and the effectiveness of drugs. Drug carriers are used in drug-delivery systems such as the controlled-release technology to prolong in vivo drug actions, decrease drug metabolism, and reduce drug toxicity. Carriers are also used in designs to increase the effectiveness of drug delivery to the target sites of pharmacological actions. An “adjuvant” is a substance added to a drug product formulation that affects the action of the active ingredient in a predictable way. A “vehicle” is an excipient or a substance, preferably without therapeutic action, used as a medium to give bulk for the administration of medicines. Such pharmaceutical carriers, adjuvants or vehicles can be sterile liquids, such as water and oils, including those of petroleum, animal, vegetable or synthetic origin, such as peanut oil, soybean oil, mineral oil, sesame oil and the like, excipients, wetting agents or diluents. Suitable pharmaceutical carriers are described in “Remington's Pharmaceutical Sciences” by E. W. Martin.

Examples of pharmaceutical compositions include any solid (tablets, pills, lozenge, powder, pellet, capsules, granules etc.) or liquid (solutions, suspensions or emulsions) composition for oral, topical or parenteral administration, among others.

Pharmaceutical dosage forms include but are not limited to parenteral preparations (such as injections, powders for injections, implants, etc.), liquid preparations for oral use (such us syrups, solutions, suspensions, emulsions, powders and granules for suspension and for solution, oral drops, etc.), oromucosal preparations (such as lozenges, sublingual and buccal tablets, oromucosal drops and sprays, etc.) solid preparations for oral use (oral powders, effervescent powders, tablets—uncoated, coated, effervescent, soluble, dispersible, orodispersible, modified release, gastro-resistant-, oral lyophilizates, capsules—hard, soft, modified release, gastro-resistant-, granules—coated, effervescent, modified release, gastro-resistant-), transdermal patches, powders for inhalation, nasal preparations and rectal preparations.

In certain embodiments, the pharmaceutical compositions are in oral form because of the convenience for the subject to be treated. Said oral pharmaceutical compositions may contain conventional excipients known in the art including binders, such as maize starch, pregelatinized maize starch, povidone, gelatine, etc.; diluents or fillers, such as microcrystalline cellulose, lactose, sodium phosphate, calcium phosphate dibasic dihydrate, calcium phosphate dibasic anhydrous, etc.; disintegrants, such as sodium croscarmellose, sodium starch glycolate, cross-linked povidone, gums, etc.; glidants, such as talc or colloidal silica; lubricants, such as magnesium stearate, stearic acid, sodium stearyl fumarate, etc.; film-formers, such as hydroxypropylcellulose, Hypromellose, hydroxy-propylmethylcellulose, etc.; opacifiers, such as titanium dioxide; coloring agents, such as sunset yellow, iron oxides, indigo carmine, erythrosine, etc.; plasticizers, such as polyethylene glycol, triacetin, etc.; acidifying agents, such as citric acid; buffering agents, such as citric acid and sodium citrate; sweetening agents, such as sucralose, aspartame, acesulfame, sodium saccharine, etc.; flavoring agents, such as strawberry flavor, lemon flavor, cola flavor, orange flavor, etc.; thickening or stabilizers such as modified celluloses (hydroxypropylcellulose, carboxymethylcellulose sodium), povidones, gums, etc.; antimicrobial and solvent agents, such as ethanol, propyleneglycol, etc.; antimicrobial preservatives, such as sodium benzoate, potassium sorbate; coloring agents, such as tartrazine, curcumin, quinoline yellow, sunset yellow, etc.; and lubricants, such as talc, magnesium stearate, stearic acid, sodium stearyl fumarate, polyethylene glycols, etc.

Solid compositions may be prepared by conventional methods of blending, filling or tableting. Repeated blending operations may be used to distribute the active agent throughout those compositions employing 1a rge quantities of fillers. Such operations are conventional in the art. Tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, e.g., antimicrobial preservatives, such as methylparaben, propylparaben, etc.; antioxidants, such as sodium metabisulfite, propyl gallate, etc.; stabilizing and suspending agents, such as soluble or swellable modified celluloses, e.g., carboxymethylcellulose sodium; tonicity agents, such as sodium chloride; and solubilizers, such as propylene glycol or polyethylene glycols.

According to some embodiments, the pharmaceutical composition may further include a therapeutically effective amount of one or more conventional agents used for the treatment and/or prophylaxis of an infection including, but are not limited to, antibacterial, antiviral or antifungal agents. Examples of antibacterial agents that can be used in combination with one or more compounds of this invention include, but are not limited to, penicillin, a cephalosporin, a carbapenem, a β-lactamase inhibitor, an aminoglycoside, an aminocyclitol, a quinolone, a macrolide, a tetracycline, a glycopeptide, a lipopeptide, a lincosamide, a streptogramin, a sulfonamide, a trimethoprim, a protein antibiotic other than said peptide, a chloramphenicol, a metronidazole, a rifampin, a fosfomycin, a methenamine, an ethambutol and a pentamidine. Examples of antiviral agents of use in combination with one or more compounds of this invention include, but are not limited to, acyclovir, a DNA synthesis inhibitor, a reverse transcriptase inhibitor, a protease inhibitor, IFN-α, and ribavirin. Examples of antifungal agents of use in combination with one or more compounds of this invention include, but are not limited to, a polyene, an imidazole, a triazole, and a glucan synthesis inhibitor.

Having demonstrated that the compounds herein exhibit antimicrobial activity, this invention also provides methods for using one or more compounds of this invention, i.e., compounds of Formula II, for inhibiting the replication of a microorganism and/or for therapeutically or prophylactically treating a microbial infection. Such methods involve contacting a microorganism with an effective amount of a compound of Formula II or pharmaceutically acceptable salt thereof so that replication of the microorganism is inhibited. When provided for therapeutic or prophylactic applications, the one or more compounds may be administered in the form of a pharmaceutical composition to a subject in need of treatment, i.e., a subject having or at risk of having a microbial infection.

Generally, an “effective amount” of the compound of the invention or a pharmaceutical composition thereof will depend on the relative efficacy of the compound chosen, the infection and/or severity of the infection being treated and the weight of the sufferer. However, active compounds will typically be administered once or more times a day for example 1, 2, 3 or 4 times daily, with typical total daily doses in the range of from 0.1 to 1000 mg/kg/day.

The term “treatment” or “to treat” in the context of this invention means administration of a compound or formulation according to the invention to prevent, ameliorate or eliminate the disease or one or more symptoms associated with said disease. “Treatment” also encompasses preventing, ameliorating or eliminating the physiological sequelae of the disease. The term “ameliorate” is understood as meaning any improvement in the situation of the subject that has been treated, either subjectively (feeling of the subject) or objectively (measured parameters).

For the purposes of this invention, a microorganism refers to a bacterium, fungus or virus. In some aspects, the microorganism is a mammalian pathogen. For example, the microorganism can be, e.g., a Gram positive bacterium, a Gram negative bacterium, a fungus, a yeast, a virus, or even a lipid enveloped virus. Particularly relevant bacteria are those of the genera Staphylococcus, Streptococcus, Pseudomonas, Enterococcus, and Escherichia. Relevant viruses include, but are not limited to RNA viruses such as Orthomyxoviruses, Ebola virus, influenza virus, Hepatitis C virus, coronavirus, West Nile virus, Vesicular Stomatitis Virus and retroviruses, e.g., human T-cell lymphotropic virus and human immunodeficiency virus; or DNA viruses such as adenoviruses, herpes virus, papillomaviruses, poxviruses, parvoviruses, and hepadnaviruses. Relevant fungi are those of the genera Aspergillus, Fusarium, and Candida. Exemplary microorganisms include, e.g., Staphylococcus aureus (including resistant strains such as methicillin-resistant Staphylococcus aureus (MRSA)), Staphylococcus epidermidis, Streptococcus pneumoniae, Enterococcus faecalis, Escherichia coli, Aspergillus niger, Aspergillus fumigatus, Candida albicans, Candida glabrata, and Candida krusei. In particular aspects, the microorganism is a virus or bacterium. More particularly, the microorganism is a viral or bacterial pathogen of mammals, in particular humans.

The following non-limiting examples are provided to further illustrate the present invention.

### Example 1: Synthesis of Unsymmetrical Bismuthanes

The first unsymmetrical bismuthanes (Ar¹)₂(Ar²)Bi were prepared with naphthyliodide (Gilman & Yablunky (1941) J. Am. Chem. Soc. 63(1):207-211; Gilman & Yablunky (1941) J. Am. Chem. Soc. 63(1):212-216). However, the main issue in this work was the limited substrate scope. The vast majority of the prepared compounds were naphthyl derivatives. Naphthyl derivatives are likely less prone to dismutation (the scramble process) due to the increased size of the naphthyl substituent relative to phenyl (Matano, et al. (1992) Bull. Chem. Soc. Jpn. 65(12):3504-3506). Beside naphthyl derivatives, only three non-naphthyl derivatives were prepared in these early studies, specifically (p-tolyl)₂(p-ClC₆H₄)Bi, (p-ClC₆H₄)₂(o-CH₃C₆H₄)Bi, Ph₂(p-ClC₆H₄)Bi in 55%, 45% and 33% yield, respectively. The yields were diminished likely due to the dismutation which creates mixtures difficult to separate (hence low yields and limited scope).

Other methods for preparation of Ar¹₂Ar²Bi have also been described. In particular, Ar₂BiOTf (Tf triflate) stabilized with HMPA (cancerogen) has been described (Matano, et al. (1996) Organometallics 15(7):1951-1953), as has the use of symmetrical Ar₃Bi (Matano, et al. (2001) Synthesis 2001(07):1081-1085). However, the latter method requires seven steps to produce the final product.

The present invention focuses on comparison of the ate-complex-promoted and the electrophilic modes of dismutation, how to suppress them, and then uses the information to enhance Bi—C bond formation leading to an efficient and streamlined synthesis of electronically diverse unsymmetrical organobismuthanes Ar¹₂Ar²Bi (1). These compounds (1) can now be prepared through a simple two-step procedure that is no longer limited by reagent scope (Suzuki & Murafuji (1992) J. Chem. Soc. Chem. Commun. 1143-1144; Matano, et al. (1996) Organometallics 15(7):1951-1953) or multistep processes (Matano, et al. (2001) Synthesis 2001(07):1081-1085).

First, a systematic study of the effect of the type of nucleophile and electrophile on the formation of the unsymmetrical bismuthane 1 (Ar¹₂Ar²Bi) and the related dismutated products (Ar²₂Ar¹Bi, Ar¹₃Bi, and Ar²₃Bi) was investigated. Our reaction of choice utilized diphenylbismuth halide or tosylate, Ph₂BiX, as the electrophilic source and an organometallic nucleophile prepared from 4-bromobenzaldehyde dimethyl acetal affording easily separable diphenyl-(dimethoxymethylphenyl)bismuthane (1a) from the main dismutated side product, triphenylbismuthane (Ph₃Bi). The ratio of products served as a measure of the dismutation process (Scheme 1).

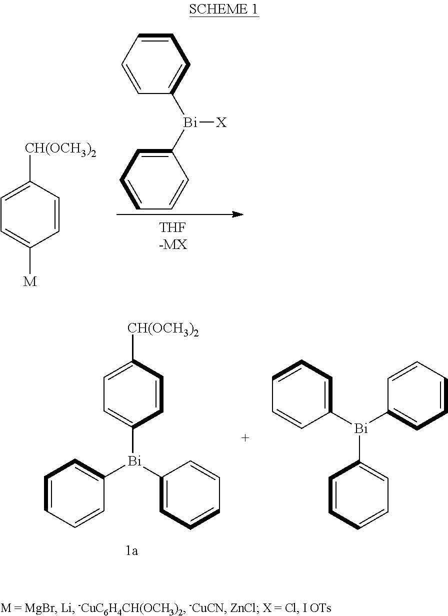

Another advantage of this model system for testing various nucleophiles and electrophiles in Bi—C bond-forming reactions is the relatively similar electronic properties of these two aryl groups (Ar¹=Ph and Ar²=C₆H₄CH(OCH₃)₂), limiting the electronic influence on Bi—C bond formation process.

Dismutation was evaluated by varying the nucleophilic sources (Table 1, entries 1-6) from hard nucleophiles, operating often through a single electron transfer mechanism to soft nucleophiles operating by an ionic mechanism (Johnson & Dutra (1973) J. Am. Chem. Soc. 95(23):7783-7788).

TABLE 1

| Entry | Reagent | Ph₂BiX | 1a^{a} | BiPh₃^{a} |
|---|---|---|---|---|
| 1 | ArMgBr | Ph₂BiCl | 30% | 30% |
| 2 | ArLi | Ph₂BiCl | 40% | 9% |
| 3 | [Ar₂Cu]MgX | Ph₂BiCl | 64% | 4% |
| 4 | [Ar₂Cu] Li | Ph₂BiCl | 59% | 7% |
| 5 | [Ar₂CuCN] MgX | Ph₂BiCl | 19% | 23% |
| 6 | ArZnX | Ph₂BiCl | 74% | 12% |
| 7 | ArMgBr | Ph₂BiI | 25% | 15% |
| 8 | ArLi | Ph₂BiI | 4% | 13% |
| 9 | [Ar₂Cu] MgX | Ph₂BiI | 32% | 14% |
| 10 | [Ar₂Cu] Li | Ph₂BiI | 64% | 17% |
| 11 | ArZnX | Ph₂BiI | 74% | 7% |
| 12 | ArMgBr | Ph₂BiOTs | 92% | <1% |
| 13 | ArLi | Ph₂BiOTs | 21% | 15% |
| 14 | [Ar₂Cu] MgX | Ph₂BiOTs | 95% | 1% |
| 15 | [Ar₂Cu] Li | Ph₂BiOTs | 40% | 4% |
| 16 | ArZnX | Ph₂BiOTs | 94% | 0% |

^{a}Isolated yields of triarylbismuthane 1a and Ph₃Bi from the model reaction in Scheme 1. Typical reaction conditions: THF, −10° C., 1 hour, 40 minutes. Details are provided in Example 2.

All the nucleophiles were compared to a single electrophile, diphenylbismuth chloride, Ph₂BiCl. Grignard reagents (entry 1) showed a rather poor yield of the product 1a (30%) accompanied by a relatively high amount of the Ph₃Bi (30%). Organolithiums (entry 2) only slightly increased the yield of 1a to 40% while significantly decreasing the amount of Ph₃Bi to 9%. Organocuprates (entries 3-5), prepared from a Grignard reagent (entry 3) or an organolithium (entry 4), showed improved yields of 1a 64% and 59% and decreased the formation of the triphenylbismuthane to 4% and 7%, respectively. Surprisingly, the cyanocuprate formed from the Grignard reagent (entry 5) showed a poor yield of 1a (19%), and increased the amount of triphenylbismuthane (23%). The highest yield was achieved with an organozinc (entry 6), affording 1a in 74% yield with a somewhat elevated amount of the Ph₃Bi (12%).

In entries 7-16, the electrophile was varied. In comparison with diphenylbismuth chloride, diphenylbismuth iodide (entries 7-11) showed a somewhat similar trend, with the best result achieved by the organozinc nucleophile (entry 11) reaching the same yield of 1a (74%) as with Ph₂BiCl, reducing formation of triphenylbismuth to 7%. The best-performing electrophile turned out to be diphenylbismuth tosylate, Ph₂BiOTs (2a), which afforded poor and moderate yields with organolithium (entry 13) and lithium diorganocuprates (entry 15), respectively, but excellent yields (exceeding 90%) with Grignard (entry 12, 92%), magnesium diorganocuprate (entry 14, 95%), and organozinc (entry 16, 94%) reagents. For further experiments, organozinc reagents, which demonstrated virtually no dismutation, were selected. Moreover, organozinc displayed better functional group tolerance in comparison with Grignard reagents, consuming less starting material in comparison with diorganocuprate reagents that utilize only one out of two aryl groups. These results indicate that the selection of the electrophile is more significant for a selective synthesis of heteroleptic triarylbismuthanes than the selection of the nucleophile.

Next, a comparison of nucleophilic and electrophilic modes of dismutation by the addition of either a sub-stoichiometric amount of PhMgBr, PhZnX (X=Br or Cl), MgCl₂, ZnCl₂, Ph₂BiCl, Ph₂BiI, or Ph₂BiOTs to diphenyl(4-methoxyphenyl)-bismuthane 1b was explored (Scheme 2).

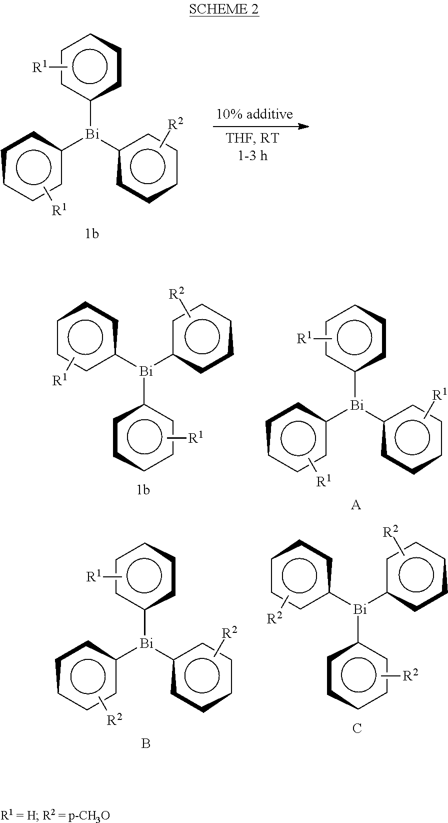

Bismuthane 1b was selected given that an electron-rich group is a better leaving group than an electron-poor one (Bras, et al. (1983) J. Organomet. Chem. 256(1):C₁-C4), making 1b an ideal substrate for studying dismutation of triarylbismuthanes. The results of this analysis are presented in Table 2.

TABLE 2

| Entry | Additive | 1b [%] ^{a} | 1b:A:B:C^{b} |
|---|---|---|---|
| 1 | PhMgBr | 99 | — |
| 2 | PhZnX | 98 | — |
| 3 | MgCl₂ | 95 | — |
| 4 | ZnCl₂ | 89 | — |
| 5 | Ph₂BiCl | 30 | 11.3:9.2:5.7:1 |
| 6 | Ph₂BiI | 43 | 17.5:15.1:5.6:1 |
| 7 | Ph₂BiOTs | 33 | 18.6:15.1:3.7:1 |

^{a} Recovered material.
^{b}Molar ratio.

Surprisingly, the Grignard reagent (entry 1), organozinc (entry 2), MgCl₂ (entry 3), and ZnCl₂ (entry 4) had no effect within the time period, and 1b was recovered in essentially quantitative yields. However, the bismuth-based electrophiles, Ph₂BiCl (entry 5), Ph₂BiI (entry 6), and Ph₂BiOTs (entry 7) decreased the amount of recovered 1b to 30%, 43%, and 33% and afforded a mixture of three additional dismutated products A, B, and C in a molar ratios of 11.3:9.2:5.7:1, 17.5:15.1:5.6:1, and 18.6:15.1:3.7:1 of 1b/A/B/C after 1 hour at room temperature in tetrahydrofuran (THF), respectively. Subsequent experiments with Ph₂BiI demonstrated that the time frame for equilibration is variable, sometimes taking up to 6 hours. This induction period may indicate that a secondary reagent or process derived from the reaction or decay of Ph₂BiI might be responsible for the dismutation. Nevertheless, these results indicate that the ate-complex-intermediate mechanism is accessible when the nucleophile is in excess but only at higher temperatures (reflux in diethyl ether) and elongated reaction times (20 hours; Gilman, et al. (1939) J. Am. Chem. Soc. 61(5):1170-1172).

In general, the dismutation is far more likely when triggered by the electrophilic Ph₂BiX, and it occurs at ambient temperatures and shorter reaction times (1-6 hours). Therefore, keeping the concentration of the electrophile to a minimum, achieved by slow addition of the electrophile into a solution of nucleophile, rather than the previously reported reversed order of the reagents (Kauffmann, et al. (1985) Chem. Ber. 118(3):1031-8) was posited to obtain high yields of heteroleptic triarylbismuthanes. To support this, two experiments were conducted which differed in the order of the reagent addition. In the first experiment, 1 equiv of nucleophile p-CH₃OC₆H₄ZnX was added in four portions into 1 equiv of electrophile Ph₂BiI over 2 hours at room temperature affording the isolated yield of 1b in 31% with the ratio of 1b/A/B being 2.8:2.1:1. In the second experiment, four portions of the electrophile were added into the nucleophile under the same conditions providing an isolated yield of 1b in 55% with the ratio of 1b/A/B being 13.9:0.85:1 (see Example 2 for more details). This result is in agreement with the results of the reaction in Scheme 2 and indicates that the order of addition of reactants is important.

To demonstrate the generality and applicability of these findings, a set of diverse unsymmetrical triarylbismuthanes, Ar¹₂Ar²Bi, 1a-1h (Table 3), was targeted through a two-step process (Scheme 3A and Scheme 3B).

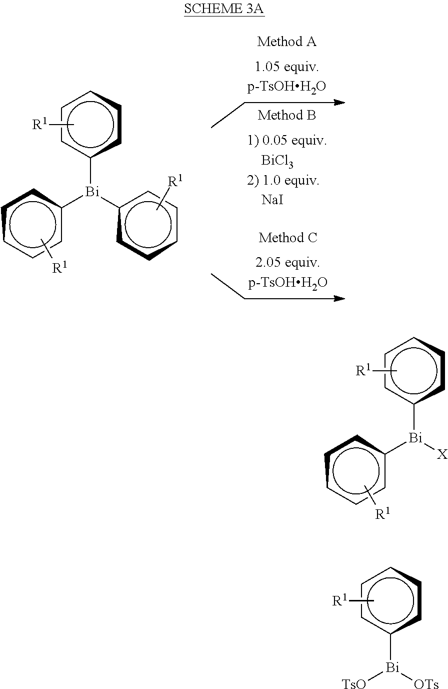

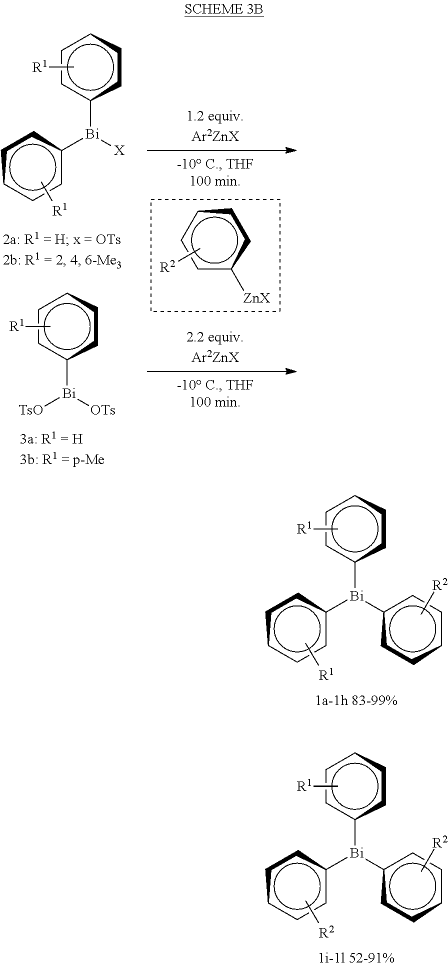

TABLE 3

| Method A Compounds | Method B Compounds | Method C Compounds |
|---|---|---|
| 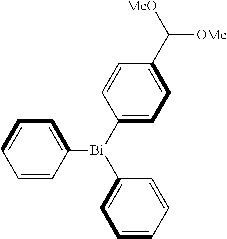 1a 94% | 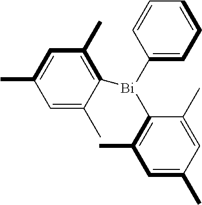 1f 94% | 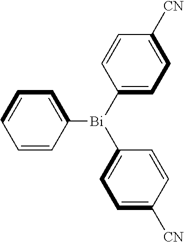 1i 87% |
| 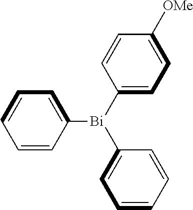 1b 99% | 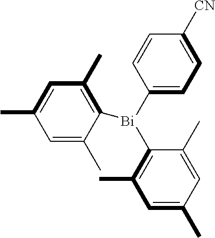 1g 92% | 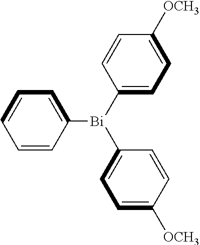 1j 91% |
| 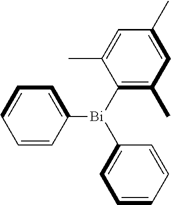 1c 97% | 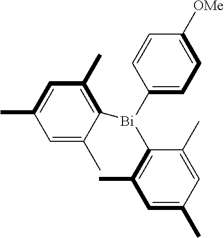 1h 94% | 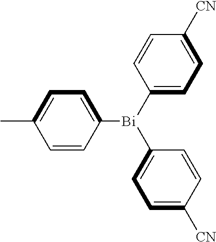 1k 87% |
| 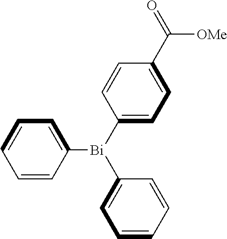 1d 83% | | 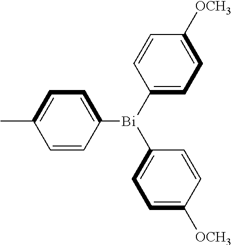 1l 52% |
| 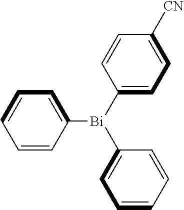 1e 84% | | |

Overall, bismuthanes 1 were formed in good to high yields (83-99%) regardless of the electronic properties of their substituents. In the first step, previously reported diphenylbismuth tosylate (2a) (Scheme 3A, Method A) was prepared from a simple combination of triphenylbismuthane with a slight excess of p-toluenesulfonic acid (p-TsOH·H₂O) in diethyl ether (Deacon, et al. (2986) Inorg. Chim. Acta 113(1):43-6). An attempt was made to synthesize other diarylbismuth tosylates from the corresponding triarylbismuthanes, such as Bi(p-CH₃OC₆H₄)₃, Bi(p-NCC₆H₄)₃, and trimesitylbismuthane. However, it was difficult to isolate them in sufficient purity; inseparable mixtures of the desired products Ar₂BiOTs, starting materials Ar₃Bi, and arylbismuth ditosylates ArBi(OTs)₂ were often obtained. Although these impure species could be used in the following step, the yield was diminished, and the corresponding product needed to be isolated from a complicated reaction mixture (see Example 2 for more details). As stated previously, increased steric congestion retards the dismutation process; therefore, dimesitylbismuth iodide (Matano, et al. (1992) Bull. Chem. Soc. Jpn. 65(12):3504-6), Mes₂BiI (2b), prepared by treatment of trimesitylbismuthane with BiCl₃ followed by halide exchange with sodium iodide (Scheme 3A, Method B), afforded comparable results to the monotosylate 2a with minimal formation of the dismutated byproducts (vide infra). In the second step, diarylbismuth tosylate or iodide 2 was added to 1.2 equiv of organozinc reagent, Ar₂ZnX, at −10° C., and the resulting reaction mixtures were stirred for 100 minutes, affording the target set of products 1 in favorable yields without formation of the dismutated byproducts. Notably, when the products were formed in a high yield, there is no dismutation and the product could be easily isolated. This was especially important in cases where substituents were chemically similar, for example Ph₂MesitylBi (1c) vs PhMesityl₂Bi (1f). These substrates would be difficult to separate if formed in the same reaction mixture due to close polarity or similar physical properties.

An alternative approach was also explored wherein utilizing monoarylbismuth ditosylates 3, prepared from the corresponding homoleptic triarylbismuthane and 2 equiv of p-TsOH·H₂O under reflux in high yields (Scheme 3A, Method C). In the following step, compound 3 was treated with the organozinc reagent yielding the corresponding heteroleptic triarylbismuthanes 1i-1l. However, in comparison with the previous protocols, the yields obtained for 1i-1l were fairly low (52-91%) relative to the first two synthetic protocols accessed through diarylbismuth tosylates 2. To unambiguously confirm the structure of the heteroleptic triarylbismuthanes 1, an X-ray of 1k was obtained. The bond lengths and angles closely match the parent triphenylbismuthane (Berger, et al. (2012) Phys. Chem. Chem. Phys. 14(44):15520-4).

Factors responsible for the dismutation of heteroleptic triarylbismuthanes, which had previously obscured their syntheses, were subsequently investigated. In accordance with the instant method, the selection of the electrophile is important, improving efficiency by minimizing dismutation, which otherwise diminishes the yield and purity of the product. When the bismuth electrophiles were compared, the best results were obtained with diphenylbismuth tosylate (2a), which afforded the highest yields of product 1 with virtually no formation of byproducts. The superior performance of 2a over diphenylbismuth halides may result from the chelate-like coordination of the tosylate ligand to the organobismuth counterpart, or its inability to form a bridged species, thus increasing its stability to dismutation. Alternatively, it may reduce solubility, effectively shortening the time the reactive species exists in solution. The soft or hard nature of the nucleophile does not seem to have much of an effect, since the Grignard and organozinc reagents give comparable yields and amounts of dismutated byproducts. Steric congestion around the bismuth center significantly reduces dismutation, which can be demonstrated by better performance in yield and selectivity of dimesitylbismuth iodide (2b) versus Ph₂BiI. A second important observation according to this study (Scheme 2) is maintaining a minimal concentration of the bismuth electrophile during the course of the reaction. This was accomplished by adding the electrophile to a solution of the nucleophile. These findings permit an efficient two-step synthetic protocol utilizing either diarylbismuth or monoaryl precursors and afford an electronically diverse set of heteroleptic triarylbismuthanes 1, Ar¹₂Ar²Bi, without the formation of dismutated contaminants.

### Example 2: Materials and Methods

General Comments on Experimental Section. All reactions were carried out under nitrogen atmosphere. All reagents and solvents were purchased from commercial suppliers. BiCl₃ and ZnCl₂ were purified by reflux with thionyl chloride, then dissolved in diethyl ether and filtered to remove impurities. p-Toluene-sulfonic acid monohydrate (p-TsOH·H2O) was purified by co-distillation in toluene. All other reagents were used as purchased without further purification. Solvents were purified through an alumina column solvent system and further dried with molecular sieves. Column chromatography was performed with 35-70 mesh silica gel using flash column techniques or COMBIFLASH® NextGen System. Varian UNITY INOVA™ 500 MHz was used for recording the ¹H and ¹³C NMR spectra. Chemical shifts for ¹H and ¹³C and were given in part per million (ppm), referenced internally according to the residual solvent resonances. Coupling constants were given in Hertz (Hz) and the following abbreviations were used: s, singlet; d, doublet; t, triplet; m, multiplet.

Diphenylbismuth chloride (Ph₂BiCl), diphenylbismuth iodide (Ph₂BiI) and dimesitylbismuth iodide 2b were prepared according to previously reported procedures (Barton, et al. (1986) Tetrahedron 42(12):3111-3122; Matano, et al. (19920 Bull. Chem. Soc. Jpn. 65(12):3504-6).

Instrumentation. ¹HNMR and ¹³CNMR were collected on a Varian UNITY INOVA™ 500 MHz. EA samples were analyzed with a PERKINELMER® 2400 Series II Analyzer. HPLC chromatograms were collected on an AGILENT® 1200 series using a 25 minute 25:75 to 0:100 H₂O :Acetonitrile gradient on a DISCOVERY® C_{18 25} cm×4 mm, 5 μm column.

Diphenylbismuth tosylate (2a). The synthetic protocol was modified from a previously reported procedure (Deacon, et al. (1986) Inorg. Chim. Acta 113:43-46). To a diethyl ether solution of triphenylbismuthane (20.0 g, 45.4 mmol) was added dropwise a solution of p-TsOH·H2O (8.64 g, 45.4 mmol) in diethyl ether. The reaction mixture was allowed to stir for 5-6 hours, then filtered and the collected solid washed two times with diethyl ether to afford 20.8 g, 86% 2a .

Phenylbismuth ditosylate (3a). The protocol was modified from a previously reported procedure (Deacon, et al. (1986) Inorg. Chim. Acta 113:43-46). To a diethyl ether solution of triphenylbismuthane (1.0 g, 2.3 mmol in 10 ml) was added dropwise a solution of p-TsOH·H₂O (0.91 g, 4.7 mmol). The reaction mixture was stirred at 90° C. for 3 hours, then cooled to room temperature, filtered, and the collected solid washed two times with diethyl ether (40 ml) affording quantitatively phenylbismuth ditosylate (3a).

p-Tolylbismuth ditosylate (3b).

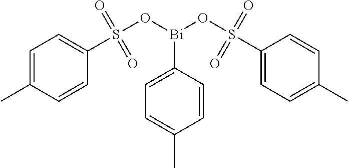

A solution of p-TsOH·H₂O (2.22 g, 11.7 mmol) was added slowly to a solution of tri(p-tolyl)bismuthane (2.77 g, 5.75 mmol) in diethyl ether. The reaction mixture stirred at 90° C. for 5 hours, then cooled to room temperature, filtered, and the collected solid washed two times with diethylether (25 ml) affording the product (3.65 g, 5.69 mmol) in 99% yield.

White Powder. ¹H NMR (d₆-DMSO): δ 8.57 (d, J=7.6 Hz, 2H, C₆H₄—CH₃, CH), 7.83 (d, J=7.6 Hz, 2H, C₆H₄—CH₃, CH), 7.48 (d, J=7.7 Hz, 4H, ArSO₃Bi, CH), 7.11 (d, J=7.7 Hz, 4H, ArSO₃Bi, CH), 2.28 (overlapping singlets, 9H, C₆H₄—CH₃). ¹³C NMR (d₆-DMSO): δ 145.8, 138.2, 137.2, 136.8 (Ar—Bi, CH), 134.0 (Ar—Bi, CH), 132.7, 128.5 (ArSO₃Bi, CH), 125.9 (ArSO₃Bi, CH), 21.9 (C₆H₄—CH₃), 21.2 (C₆H₄—CH₃). Anal. Calc. for BiO₆C₂₁H₂₁S₂: C, 39.26; H, 3.29. Found: C, 38.87; H, 2.87. 3b did not pass EA in % H. EA was consistently low in % C and % H.

ArLi. 4-bromobenzaldehyde dimethylacetal (260 mg, 1.12 mmol) was added to a Schlenk flask and dissolved in 5 ml THF, then cooled to −78° C. A solution of n-butyllithium in hexanes (1.12 mmol, 0.7 ml) was added dropwise to the reaction solution and stirred at −78° C. for 40 minutes. Then, diphenylbismuth tosylate (0.936 mmol) was added and the reaction mixture was allowed to warm to −10° C. The final concentration was kept to 0.075 M of the nucleophile in THF. The reaction was stirred for 1 hour and 40 minutes. Afterwards, the reaction mixture was allowed to warm to ambient temperature.

Subsequently, the reaction mixture was quenched with a saturated solution of NaHCO₃ in distilled water and diluted with EtOAc (15 ml), upon which the two phases were separated. The aqueous phase was washed twice with EtOAc (10 ml). The combined organic phases were washed twice with sat. NaHCO₃ (10 ml) and twice with sat. brine solution (10 ml). Finally, the organic phase was dried over MgSO₄, filtered through silica gel, and concentrated in vacuo. The crude reaction mixture was then purified by column chromatography using a 20:1 hexanes:ethyl acetate eluent.

[Ar₂Cu]Li. To a freshly prepared solution of 4-lithium benzaldehyde dimethyl acetal solution (2.25 mmol, 0.075 M) at −78° C. was added CuI (1.12 mmol) and the solution was warmed to −40° C. and stirred for 1 hour. To this organocuprate reagent was added diphenyl bismuth tosylate (0.936 mmol) and warmed to −10° C. The final concentration was kept to 0.075 M of the nucleophile in THF. The reaction was stirred for 1 hour and 40 minutes. Afterwards, the reaction was worked up as previously described.

ArMgBr. A modified preparation of Grignard reagent was used to prepare a stock solution of organomagnesium reagent. Magnesium turnings (7.9 g, 325 mmol) were added to a Schlenk bomb and to it was added 100 ml THF. 4-Bromobenzaldehyde dimethylacetal (130 mmol) was dissolved in THF (50 ml) and added dropwise to the magnesium. The mixture was allowed to stir for 30 minutes, then heated to 65° C. for 4 hours. A solution of organomagnesium reagent (1.12 mmol) was cooled to −10° C. To this solution was added diphenylbismuth tosylate (0.936 mmol) to give a final concentration of 0.075 M of the nucleophile in THF. The reaction was stirred for 1 hour and 40 minutes. Afterwards, the reaction was worked up as previously described.

[Ar₂Cu]MgX. A solution of Grignard reagent (2.25 mmol) was cooled to −40° C. and to it was added CuI (1.12 mmol) and stirred for one hour. To this organocuprate reagent was added diphenylbismuth tosylate (0.936 mmol) to give a final concentration of 0.075 M of the nucleophile in THF. The reaction was warmed to −10° C. and stirred for 1 hour and 40 minutes. Afterwards, the reaction was worked up the same as previously described.

[ArCuCN]MgX. A solution of organomagnesium reagent (1.12 mmol) was cooled to −40° C. and to it was added CuCN (1.12 mmol) and stirred for one hour. To this organocuprate reagent was added diphenylbismuth tosylate (0.936 mmol) to give a final concentration of 0.075 M of the nucleophile in THF. The reaction was warmed to −10° C. and stirred for 1 hour and 40 minutes. Afterwards, the reaction was worked up the same as previously described.

ArZnX. To a solution of Grignard reagent (1.12 mmol) was added anhydrous ZnCl₂ (153 mg, 1.12 mmol) dissolved in 5 ml THF. The reaction was stirred at room temperature for about 10 minutes, then cooled to −10° C. Next, diphenylbismuth tosylate (0.936 mmol) was added to the reaction mixture to give a final concentration of 0.075 M of the nucleophile in THF. The reaction was allowed to stir at −10° C. for 1 hour and 40 minutes. Afterwards, the reaction was worked up the same as previously described.

Investigation of Dismutation Initiated by 10 mol % Additive (Scheme 2). To a stirring THF (2 mL) solution of diphenyl(4-methoxylphenyl)bismuthane 1b, (100 mg, 0.213 mmol), a THF (1 mL) solution of 10 mol % additive (0.0213 mmol) was added and stirred for one hour. After one hour, the mixture was filtered through Diatomaceous earth sold under the tradename CELITE® and THF was removed in vacuo and the products were separated by Combiflash. The isolated yields of Ph₂BiAnisyl (1b), Ph₃Bi (A), Anisyl₂BiPh (B), Anisyl₃Bi (C) are provided in Table 4.

TABLE 4

| | | 1b | A | B | C | Recovery | 1b:A:B:C |
|---|---|---|---|---|---|---|---|
| Entry | Additive | (mg) | (mg) | (mg) | (mg) | 1b % | (molar ratios) |
| 1 | PhMgBr | 99 | — | — | — | 99 | — |
| 2 | PhZnBr | 98 | — | — | — | 98 | — |
| 3 | MgCl₂ | 95 | — | — | — | 95 | — |
| 4 | ZnCl₂ | 89 | — | — | — | 89 | — |
| 5 | PhzBiCl | 30 | 23 | 16 | 3 | 30 | 11.3:9.2:5.7:1 |
| 6 | Ph₂BiI | 43 | 35 | 14 | 2.7 | 43 | 17.5:15.1:5.6:1 |
| 7 | Ph₂BiOTs | 33 | 25 | 7 | 2 | 33 | 18.6:15.1:3.7:1 |

Investigation of Dismutation Initiated by the Slow Addition of Electrophile into Nucleophile. To a stirring THF (2 mL) solution of nucleophile, p-MeOC₆H₄ZnX (0.408 mmol), a THF (0.25 mL) solution of diphenylbismuth iodide (50 mg; 0.102 mmol), was added four times in half hour intervals to give a final volume of 3 ml and an equimolar ratio of electrophile to nucleophile. After the final addition and stirring for 30 minutes, the reaction was then quenched with sat. NaHCO₃ aqueous solution and extracted with ethyl acetate 2× 10 ml. The combined organic phase was washed with sat. NaHCO₃(2×10 ml), dried over MgSO₄, and the solvent was removed in vacuo. Finally, the crude product was purified through a silica gel plug and the products were separated by Combiflash. The isolated yields were as follows: Ph₂BiAnisyl (1b): 105 mg (0.223 mmol, 55%); Ph₃Bi (A): 6 mg (0.0136 mmol, 3%); Anisyl₂BiPh (B): 8 mg (0.0160 mmol, 4%).

Investigation of Dismutation Initiated by the Slow Addition of Nucleophile into Electrophile. To a stirring THF (2 mL) solution of diphenylbismuth iodide (200 mg; 0.408 mmol), a THF (0.25 mL) solution of nucleophile, p-MeOC₆H₄ZnX (0.102 mmol), was added four times in half hour intervals to give a final volume of 3 ml and an equimolar ratio of electrophile to nucleophile. After the final addition and stirring for 30 minutes, the reaction was then quenched with sat. NaHCO₃ aqueous solution and extracted with ethyl acetate 2× 10 ml. The combined organic phase was washed with sat. NaHCO₃(2×10 ml), dried over MgSO₄, and the solvent was removed in vacuo. Finally, the crude product was purified through a silica gel plug and the products were separated by Combiflash. The isolated yields were as follows: Ph₂BiAnisyl (1b): 60 mg (0.128 mmol, 31%); Ph₃Bi (A): 42 mg (0.0954 mmol, 23%); Anisyl₂BiPh (B): 23 mg (0.0460 mmol, 11%).

General Procedure Utilizing Organozincs and Diarylbismuth Tosylate or Iodide (2) (Methods A and B). For compounds 1a, 1c, 1e, 1f, 1h, organozinc reagent was prepared by the addition of anhydrous ZnCl₂ (153 mg, 1.12 mmol) dissolved in 5 ml THF to a solution of Grignard reagent (1.12 mmol). For compounds 1b, 1g, an organozinc reagent was prepared by Knochel's TurboGrignard procedure (Grassi, et al. (2019) Chem. Eur. J. 25:3752-55). Compound 1d was prepared using Rieke's procedures (Zhu, et al. (1991) J. Org. Chem. 56:1445-1453).

The volume of solution of organozinc reagent (1.12 mmol) was adjusted to a final volume of 15 ml by addition of dry THF. The reagent was stirred at room temperature for about 10 minutes, then cooled to −10° C. After that, diarylbismuth tosylate (Method A) or iodide (Method B) 2 (0.936 mmol) was added at once to a stirred organozinc solution. The reaction mixture was stirred for 1 hour and 40 minutes and allowed to warm to room temperature. Then, the reaction was quenched by addition of a saturated solution of NaHCO₃ in distilled water and extracted with EtOAc (15 ml). The aqueous phase was washed twice with EtOAc (10 ml). The combined organic phases were washed twice with saturated NaHCO₃ (10 ml) and twice with saturated brine solution (10 ml). Finally, the organic phase was dried over MgSO₄, filtered through silica gel and concentrated in vacuo affording crude product. Further purification or recrystallization methods are described below for each compound.

General Procedure Utilizing Organozincs and Arylbismuth Ditosylates (3) (Method C). Regarding compounds 1i and 1k, an organozinc reagent was prepared by Knochel's protocol (Grassi, et al. (2019) Chem. Eur. J. 25:3752-55). For compounds 1j and 1l, organozinc reagent was prepared by the addition of anhydrous ZnCl₂ (293 mg, 2.15 mmol) dissolved in 5 ml THF to a solution of Grignard reagent (2.06 mmol).

The volume of the organozinc reagent (2.06 mmol) was adjusted by addition of THF to give a final volume of 15 ml, then subsequently cooled to −10° C. and stirred. The monoarylbismuth ditosylate (0.936 mmol) was added at once to the stirred organozinc solution at −10° C. and the reaction mixture was stirred for 1 hour and 40 minutes and then allowed to warm to room temperature. The work up is identical to Method A. Further purification or recrystallization methods are described below for each compound.

Diphenyl(4-dimethoxymethylphenyl)bismuthane (1a).

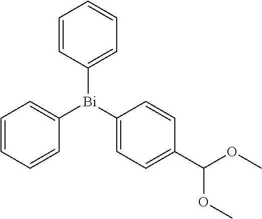

Yield: 94%. This compound was obtained according to Method A from (4-benzaldehyde dimethyl acetal)magnesium(II) bromide (1.123 mmol) and diphenyl bismuth tosylate (0.5 g, 0.936 mmol). Purified by chromatography and eluted with a gradient mixture of solvents (hexane/ethyl acetate 100/0 to 20/80).

White solid. ¹H NMR (CDCl₃): δ 7.78-7.75 (m, 6H, C₆H₅), 7.48 (d, J=7.9 Hz, 2H, C₆H₄—CH(OCH₃)₂), 7.40 (m, 4H, Ar), 7.35-7.32 (m, 2H, C₆H₅), 5.37 (s, 1H, C₆H₄—CH(OCH₃)₂, 3.35 (s, 6H, C₆H₄—CH(OCH₃)₂). ¹³C NMR (CDCl₃): δ 155.4, 155.0, 137.5, 137.5 (Ar, CH), 137.4 (C₆H₅, CH), 130.5 (Ar, CH), 128.7 (C₆H₄—CH (OCH₃)₂, CH), 127.7 (C₆H₅, CH), 103.3 (C₆H₄—CH(OCH₃)₂, 52.8 (C₆H₄—CH(OCH₃)₂). Anal. Calc. for BiO₂C₂₁H₂₁: C, 49.04; H, 4.12. Found: C, 48.76; H, 3.96.

Diphenyl(4-methoxylphenyl)bismuthane (1b).

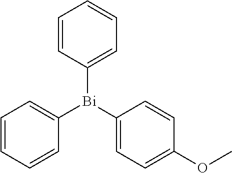

Yield: 99%. This compound was obtained according to Method A from (4-methoxyphenyl)magnesium(II) bromide (1.123 mmol) and diphenylbismuth tosylate (0.5 g, 0.936 mmol). Purified by chromatography and eluted with a gradient mixture of solvents (hexane/ethyl acetate 100/0 to 95/5.

Yellowish oil. ¹H NMR (CDCl₃): δ 7.77 (d, J=7.0 Hz, 4H, C₆H₄), 7.68 (d, J=8.5 Hz, 2H, C₆H₄—OCH₃), 7.42 (d, J=7.0 Hz, 4H, C₆H₄), 7.36-7.33 (m, 2H, C₆H₄), 6.96 (d, J=8.5 Hz, 2H, C₆H₄—OCH₃), 3.81 (s, 3H, C₆H₄—OCH₃). ¹³C NMR (CDCl₃): δ 159.3, 154.8, 145.7, 138.8 (C₆H₄—OCH₃, CH), 137.4 (Ph, CH), 130.4 (Ph, CH), 127.6 (Ph, CH), 116.4 (C₆H₄—OCH₃, CH), 55.0 (C₆H₄—OCH₃). Anal. Calc. for BiOC₁₉H₁₇: C, 48.52; H, 3.64. Found: C, 48.65; H, 3.68.

Diphenyl (2-mesityl)bismuthane (1c).

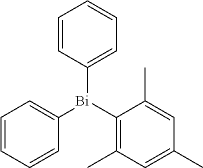

Yield: 97%. This compound was obtained according to Method A from (mesityl)magnesium(II) bromide (1.123 mmol) and diphenyl bismuth tosylate (0.5 g, 0.936 mmol). Recrystallized from cold hexanes.

White solid. ¹H NMR (CDCl₃): δ 7.86 (d, J=6.5 Hz, 4H, C₆H₅), 7.40-7.37 (m, 4H, C₆H₅), 7.32-7.29 (m, 2H, C₆H₅), 7.04 (s, 2H, C₆H₂), 2.31 (s, 3H, C₆H₂-pCH₃), 2.26 (s, 6H, C₆H₂-oCH₃). ¹³C NMR (CDCl₃): δ 157.4, 152.1, 146.2, 138.2, 137.5 (Ph, CH), 130.2 (Ph, CH), 129.3 (C₆H₂, CH), 127.4 (Ph, CH), 28.5 (C₆H₂—oCH₃), 21.1 (C₆H₂-pCH₃). Anal. Calc. for BiC₂₁H₂₁: C, 52.29; H, 4.39. Found: C, 52.19; H, 4.29.

Diphenyl(4-methoxycarbonylphenyl)bismuthane (1d).

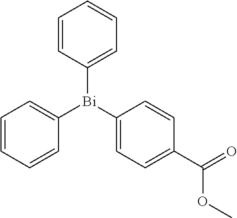

Yield: 83%. This compound was obtained according to Method A from (4-methoxycarbonylphenyl)zinc(II) halide (1.123 mmol) and diphenylbismuth tosylate (0.5 g, 0.936 mmol). Recrystallized from cold hexanes.

White solid. ¹H NMR (CDCl₃): δ 8.02 (d, J=8.1 Hz, 2H, C₆H₄—COOMe), 7.84 (d, J=8.1 Hz, 2H, C₆H₄—COOMe), 7.74 (d, J=6.6, Hz, 4H, C₆H₅), 7.43-7.40 (m, 4H, C₆H₅), 7.36-7.32 (m, 2H, C₆H₅), 3.91 (s, 3H, C₆H₄—COOCH₃, ¹³C NMR (CDCl₃): δ 167.3, 161.6, 155.3, 137.6 (C₆H₄—COOMe, CH), 137.5 (Ph, CH), 131.0 (C₆H₄—COOMe, CH), 130.6 (Ph, CH), 129.4, 127.9 (Ph, CH), 52.1 (C₆H₄—COOCH₃). Anal. Calc. for BiO₂C₂₀H₁₇: C, 48.20; H, 3.44. Found: C, 48.60; H, 3.26.

Diphenyl(4-cyanophenyl)bismuthane (1e).

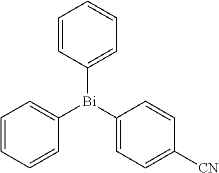

Yield: 84%. This compound was obtained according to Method A from (4-cyanophenyl)zinc(II) halide (1.123 mmol) and diphenylbismuth tosylate (0.5 g, 0.936 mmol). Purified by column chromatography and eluted with a gradient mixture of solvents (hexane/ethyl acetate 100/0 to 25/75.

White solid. ¹H NMR (CDCl₃): δ 7.87 (d, J=8.1 Hz, 2H, C₆H₄CH), 7.73 (d, J=6.6 Hz, 4H, C₆H₅), 7.60 (d, J=8.1 Hz, 2H, C₆H₄CN), 7.44 (t, J=7.3 Hz, 4H, C₆H₅), 7.37 (m, 2H, C₆H₅). ¹³C NMR (CDCl₃): δ 161.5, 155.7, 138.1 (C₆H₄CN, CH), 137.4 (Ph, CH), 133.2 (C₆H₄CN, CH), 130.8 (Ph, CH), 128.1 (Ph, CH), 119.1, 111.3. Anal. Calc. for BiNC₁₉H₁₄: C, 49.04; H, 3.03; N, 3.01. Found: C, 49.14; H, 2.96; N, 2.93.

Dimesitylphenylbismuthane (1f).

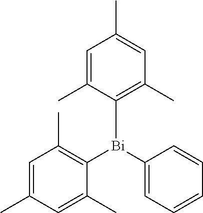

Yield: 94%. This compound was obtained according to Method B from phenylmagnesium(II) bromide (1.123 mmol) and dimesitylbismuth iodide (0.537 g, 0.936 mmol mmol). Recrystallized from hexanes.

White solid. ¹H NMR (CDCl₃): δ 7.96 (d, J=6.4 Hz, 2H, C₆H₅), 7.35-7.32 (m, 2H, C₆H₅), 7.28 (d, J=7.2 Hz, 1H, C₆H₅), 7.00 (s, 4H, C₆H₂), 2.29 (overlapping singlets, 18H, C₆H₂—CH₃). ¹³C NMR (CDCl₃): δ 155.9, 150.6, 145.5, 138.7 (Ph, CH), 137.4, 129.8 (Ph, CH), 129.2 (C₆H₂, CH), 127.1 (Ph, CH), 27.9 (C₆H₂—CH₃), 21.0 (C₆H₂—CH₃). Anal. Calc. for BiC₂₄H₂₇: C, 54.96; H, 5.19. Found: C, 54.94; H, 4.80.

Dimesityl (4-cyanophenyl)bismuthane (1g).

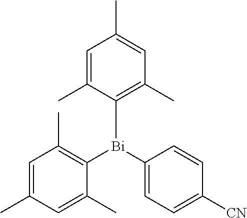

Yield: 92%. This compound was obtained according to Method B from (4-cyanophenyl)zinc(II) halide (1.123 mmol) and dimesitylbismuth iodide (0.537 g, 0.936 mmol). Recrystallized from hot ethanol.

White solid. ¹H NMR (CDCl₃): δ 8.03 (d, J=8.0 Hz, 2H, C₆H₄CN), 7.51 (d, J=8.0 Hz, 2H, C₆H₄CN), 7.01 (s, 4H, C₆H₂), 2.27 (s, 6H, C₆H₂-pCH₃), 2.22 (s, 12H, C₆H₂-oCH₃). ¹³C NMR (CDCl₃): δ 156.9, 156.6, 145.3, 139.3 (C₆H₄CN, CH), 138.0, 132.5 (C₆H₄CN, CH), 129.6 (C₆H₂, CH), 119.2, 110.7, 27.8 (C₆H₂-oCH₃), 21.0 (C₆H₂-pCH₃). Anal. Calc. for BiC₂₅H₂₆N: C, 54.65; H, 4.77; N, 2.35. Found: C, 54.83; H, 4.88; N, 2.40.

Dimesityl(4-methoxyphenylbismuthane (1h).

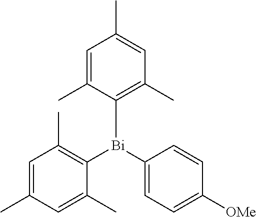

Yield: 94%. This compound was obtained according to Method B from (4-methoxyphenyl) magnesium(II) bromide (1.123 mmol) and dimesitylbismuth iodide (0.537 g, 0.936 mmol). Recrystallized from cold hexanes.

White solid. ¹H NMR (CDCl₃): δ 7.82 (d, J=8.5 Hz, 2H, C₆H₄—OCH₃), 6.98 (s, 4H, C₆H₂), 6.87 (d, J=8.5 Hz, 2H, C₆H₄—OCH₃), 3.79 (s, 3H, C₆H₄—OCH₃), 2.27 (overlapping singlets, 18H, C₆H₂—CH₃). ¹³C NMR (CDCl₃): δ 158.9, 155.5, 145.5, 140.6, 139.9 (C₆H₄—OCH₃, CH), 137.4, 129.1 (C₆H₂, CH), 115.8 (C₆H₄—OCH₃, CH), 54.9 (C₆H4-OCH3), 27.8 (C₆H₂—CH₃), 21.0 (C₆H₂—CH₃). Anal. Calc. for BiOC₂₅H₂₉: C, 54.15; H, 5.27. Found: C, 54.10; H, 5.15.

Di(4-cyanophenyl)phenylbismuthane (1i).

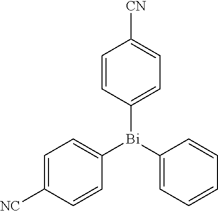

Yield: 87%. This compound was obtained according to Method C from (4-cyanophenyl)zinc(II) halide (2.06 mmol) and phenylbismuth ditosylate (0.587 g, 0.936 mmol). Recrystallized from toluene/hexanes.

White solid. ¹H NMR (CDCl₃): δ 7.84 (d, J=8.0 Hz, 4H, C₆H₄CN), 7.69 (d, J=6.5 Hz, 2H, C₆H₅), 7.64 (d, J=8.0 Hz, 4H, C₆H₄CN), 7.49-7.46 (m, 2H, C₆H₄), 7.43-7.37, (m, 1H, C₆H₅). ¹³C NMR (CDCl₃): δ 161.8, 156.3, 138.0 (C₆H₄CN, CH), 137.3 (Ph, CH), 133.5 (C₆H₄CN, CH), 131.2 (Ph, CH), 128.6 (Ph, CH), 118.8, 111.9. Anal. Calc. for BiN₂C₂₀H₁₃: C, 48.99; H, 2.67; N, 5.71. Found: C, 49.15; H, 2.62; N, 5.87.

The synthesis of this compound was attempted through Method A using phenyl magnesium bromide and bis(4-cyanophenyl)bismuth tosylate. The yield for this reaction was only 45%, as the corresponding tosylate was not obtained in analytical purity.

Di(4-methoxyphenyl)phenylbismuthane (1j).

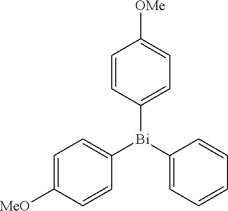

Yield: 91%. This compound was obtained according to Method C from (4-methoxyphenyl)magnesium(II) bromide (2.06 mmol) and phenylbismuth ditosylate (0.587 g, 0.936 mmol). Purified by column chromatography and eluted with a gradient mixture of solvents (hexane/ethyl acetate 100/0 to 25/75.

White solid. ¹H NMR (CDCl₃): δ 7.74 (d, J=7.5, 2H, C₆H₅), 7.64 (d, J=8.6, 4H, C₆H₄—OCH₃), 7.40-7.37 (m, J=7.5, 2H, C₆H₅), 7.34-7.31, (m, 1H, C₆H₅), 6.94, (d, J=8.6, 4H, C₆H₄—OCH₃), 3.80 (s, 6H, C₆H₄—OCH₃). ¹³C NMR (CDCl₃): 159.3, 154.6, 145.3, 138.8 (C₆H₄—OCH₃, CH), 137.4 (Ph, CH), 130.3 (Ph, CH), 127.6 (Ph, CH), 116.3 (C₆H₄—OCH₃, CH), 55.0 (C₆H₄—OCH₃). Anal. Calc. for BiO₂C₂₀H₁₉: C, 48.01; H, 3.83. Found: C, 48.24; H, 3.66.

The synthesis of this compound was attempted through Method A utilizing phenyl magnesium bromide and bis(4-methoxyphenyl)bismuth tosylate. The yield for this reaction was 87%, however the corresponding tosylate was not obtained in analytical purity.

Di (4-cyanophenyl) (p-tolyl)bismuthane (1k).

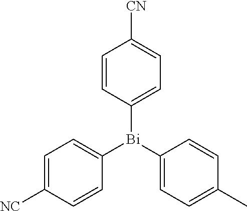

Yield: 87%. This compound was obtained according to Method C from (4-cyanophenyl)zinc(II) iodide (2.06 mmol) and (p-tolyl)bismuth ditosylate (0.547 g, 0.936 mmol). Purified by column chromatography and eluted with a gradient mixture of solvents (hexane/ethyl acetate 100/0 to 25/75.

White solid. ¹H NMR (CDCl₃): δ 7.84 (d, J=8.2 Hz, 4H, C₆H₄CN), 7.63 (d, J=8.2 Hz, 4H, C₆H₄CN), 7.57 (d, J=7.8 Hz, 2H, C₆H₄-pCH₃), 7.29 (d, J=7.8 Hz, 2H, C₆H₄-pCH₃), 2.35 (s, 3H, C₆H₄—CH₃). ¹³C NMR (CDCl₃): δ 161.6, 152.8, 138.6, 138.0 (C₆H₄CN, CH), 137.3 (C₆H₄-pCH₃, CH), 133.5 (C₆H₄CN, CH), 132.0 (C₆H₄-pCH₃, CH), 118.8, 111.7, 21.5 (C₆H₄-pCH₃). Anal. Calc. for BiN₂C₂₁H₁₅: C, 50.01; H, 3.00; N, 5.55. Found: C, 50.01; H, 2.87; N, 5.46.

Di (4-methoxyphenyl) (p-tolyl)bismuthane (1l).

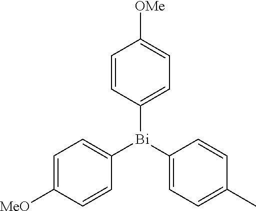

Yield: 52%. This compound was obtained according to Method C from (4-methoxyphenyl)magnesium(II) bromide (2.06 mmol) and (p-tolyl) bismuth ditosylate (0.547 g, 0.936 mmol). Purified by column chromatography and eluted with a gradient mixture of solvents (hexane/ethyl acetate 100/0 to 20/80.

White solid. ¹H NMR (CDCl₃): δ 7.65-7.61 (m, 6H, Ar), 7.20 (d, J=7.5 Hz, 2H, C₆H₄-pCH₃), 6.93 (d, J=8.4 Hz, 4H, C₆H₄—OCH₃), 3.80 (s, 6H, C₆H₄—OCH₃), 2.34 (s, 3H, C₆H₄—CH₃). ¹³C NMR (CDCl₃): δ 159.2, 150.9, 145.1, 138.7 (C₆H₄ —OCH₃, CH), 137.4 (C₆H₄-pCH₃, CH), 137.3, 131.2 (C₆H₄-pCH₃, CH), 116.3 (C₆H₄—OCH₃, CH), 55.0 (C₆H₄—OCH₃), 21.5 (C₆H₄—CH₃). Anal. Calc. for BiO₂C₂₁.H₂₁: C, 49.04; H, 4.12. Found: C, 49.06; H, 3.90.

Solved Crystal Structure of Di (4-cyanophenyl) (p-tolyl)bismuthane 1k is provided in Table 5.

TABLE 5

| | | |
|---|---|---|
| Identification Code | TLG 2-46 | |
| Empirical formula | C21 H15 Bi N2 | |
| Formula weight | 504.33 | |
| Temperature | 100.0K | |
| Wavelength | 0.71073 Å | |
| Crystal System | Triclinic | |
| Space group | P-1 | |
| Unit cell dimensions | a = 8.1718 (10) Å | α = 105.132 (4)° |
| | b = 9.9011 (14) Å | β = 105.299 (4)° |
| | c = 12.3098 (15) Å | γ = 90.697 (4)° |
| Volume | 923.9 (2) Å³ | |
| Z | 2 | |
| Density (calculated) | 1.813 Mg/m³ | |
| Absorption coefficient | 9.545 mm⁻¹ | |
| F (000) | 476 | |
| Crystal size | 0.31 × 0.29 × 0.27 mm³ | |
| Theta range for data | 1.783 to 26.425° | |
| collection | | |
| Index ranges | −10 <= h <= 8, | |
| | −12 <= k <= 12, | |
| | −15 <= l <= 15 | |
| Reflections collected | 13782 | |
| Independent reflections | 3786 [R(int) = 0.0308] | |
| Completeness to | 100.0% | |
| theta = 25.242° | | |
| Absorption correction | Semi-empirical from | |
| | equivalents | |
| Max. and Min. | 0.432 and 0.284 | |
| transmission | | |
| Refinement method | Full-matrix least- | |
| | squares on F² | |
| Data/restraints/ | 3786/21/222 | |
| parameters | | |
| Goodness-of-fit on F² | 1.380 | |
| Final R indices | R1 = 0.0350, | wR2 = 0.0659 |
| [I > 2sigma (I) ] | | |
| R indices (all data) | R1 = 0.0383, | wR2 = 0.0666 |
| Extinction coefficient | n/a | |
| Largest diff. peak and | 1.984 and −3.027 e.Å⁻³ | |
| hole | | |

### Example 3: Antibacterial Activity of Bismuth-Based Compounds

The antimicrobial activity of compounds 1-23 (Table 6) was tested to demonstrate the use of the same in treating microbial infections.

TABLE 6

| | |
|---|---|
| 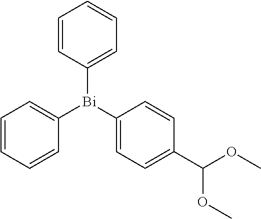 | 1 |
| 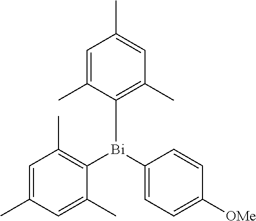 | 2 |
| 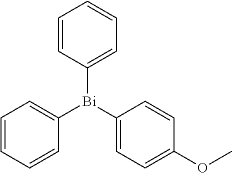 | 3 |
| 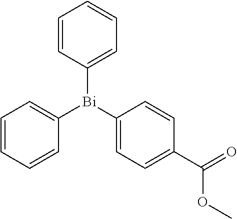 | 4 |
| 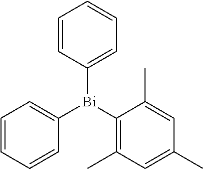 | 5 |
| 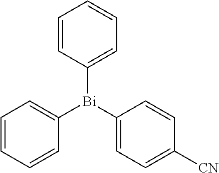 | 6 |
| 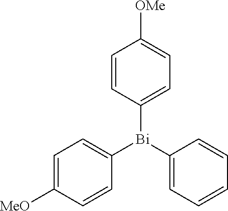 | 7 |
| 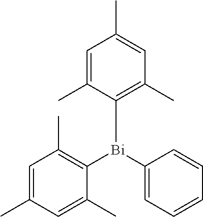 | 8 |
| 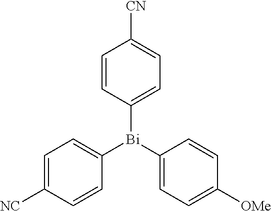 | 9 |
| 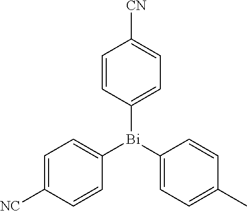 | 10 |
| 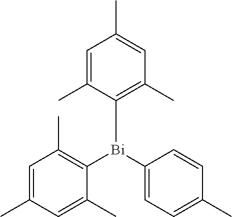 | 11 |
| 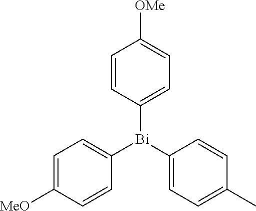 | 12 |
| 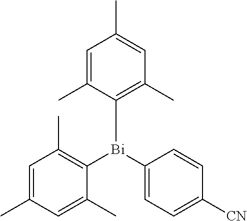 | 13 |
| 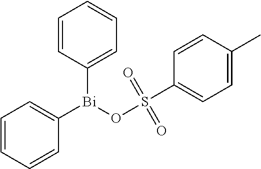 | 14 (DMSO) |
| 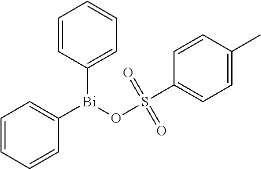 | 15 (neat) |
| 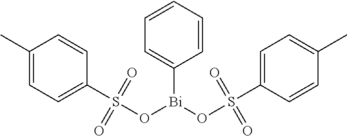 | 16 (DMSO) |
| 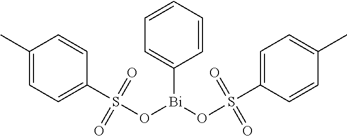 | 17 (neat) |
| 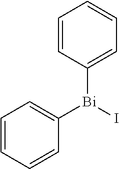 | 18 |
| 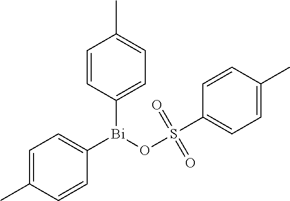 | 19 (DMSO) |
| 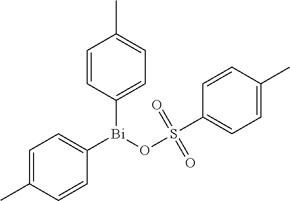 | 20 (neat) |
| 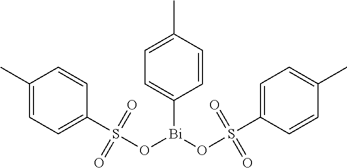 | 21 (DMSO) |
| 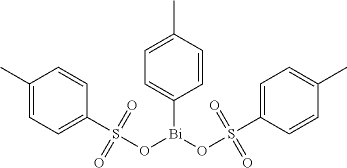 | 22 (neat) |
| 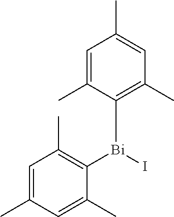 | 23 |

Compounds 14, 16, 19, 21 were provided in DMSO solution (1 mg/1 mL), and compounds 15, 17, 20, 22 were provided neat. These compounds were more prone to dismutation in solution and were therefore provided in solid form as well.

Escherichia coli and Staphylococcus aureus were streaked for colony isolation and a single colony was used to inoculate a 10 mL LB broth overnight. The following day, 250 μL of turbid broth was spread on LB agar plates. The plate was divided into four sections for a positive and negative controls plus two compounds. Positive control disks contained either 5 μg of Novobiocin (S. aureus) or 30 μg of Tetracycline (E. coli). Disks were hole punched from filter paper, autoclaved, and dipped into the respective compounds (1-5 μg compound/disk) or DMSO (negative control). The disks were placed on the plates, the plates were incubated overnight (22 hours) at 37° C. and the zone of inhibition for each compound was measured. The results of this analysis are presented in Table 7.

TABLE 7

| | Zone of Inhibition (cm) | |
|---|---|---|
| Compound | E. coli | S. aureus |
| 1 | 0 | 0 |
| 2 | 0 | 1 |
| 3 | 0 | 1 |
| 4 | 0 | 0 |
| 5 | 0 | 1.6 |
| 6 | 0 | 0 |
| 7 | 0 | 1.1 |
| 8 | 0 | 1 |
| 9 | 0 | 1 |
| 10 | 0 | 0.9 |
| 11 | 0 | 0.8 |
| 12 | 0 | 1 |
| 13 | 0 | 0.9 |
| 14 | 1.3 | 2.1 |
| 15 | — | — |
| 16 | 1 | 1.8 |
| 17 | — | — |
| 18 | 1.3 | 2.2 |
| 19 | 0.8 | 1.8 |
| 20 | — | — |
| 21 | 1 | 1.5 |
| 22 | 0 | — |
| 23 | 0.6 | 0.9-1.9 |
| DMSO | 0 | 0 |
| Novobiocin | — | 2-2.5 |
| Tetracycline | 1.8-2.5 | — |

Compounds 5, 14, 18, 19, 21 and 23 appeared to be most effective against S. aureus at the concentration tested with weaker antimicrobial activity against E. coli. In addition, compounds 2, 3, 7, 8, 9, 10, 11, 12 and 13 showed antibacterial activity against S. aureus, with little activity against E. coli at the concentration tested. Without wishing to be bound by theory, the bismuth compounds may have greater activity against gram-positive organisms.

Minimum Inhibitory Concentration (MIC) Determination. E. coli and S. aureus were grown in 96-well plates in the presence of compounds 5, 14, 16, 18, 19, and 21. For E. coli, each compound was tested at 0, 0.625, 1.25, 2.5, 5, 10, 20 and 40 μg/mL in duplicate. For S. aureus, each compound was tested at 0, 0.078, 0.157, 0.313, 0.625, 1.25, 2.5 and 5 μg/mL in duplicate. DMSO and Kanamycin (0, 0.0625, 0.125, 0.25, 0.5, 1, 2, and 4 μg/mL) were used as negative and positive controls, respectively. Plates were incubated for 24 hours at 37° C. and OD600 readings were measured to determine the minimum inhibitory concentration (MIC). The results of these analyses are presented in Table 8.

TABLE 8

| | MIC | |
|---|---|---|
| Compound | E. coli | S. aureus |
| 5 | 40 μg/mL | 0.625 μg/mL |
| 14 | 10 μg/mL | 0.625 μg/mL |
| 16 | 20 μg/mL | 0.625 μg/mL |
| 18 | 10 μg/mL | 0.625 μg/mL |
| 19 | No inhibition | 2.5 μg/mL |
| 21 | 40 μg/mL | 1.25 μg/mL L |
| DMSO | No inhibition | No inhibition |
| Kanamycin | 0.0625 μg/mL | 0.0625 μg/mL |
| DNP | — | — |

### Example 4: Antiviral Activity of Bismuth-Based Compounds

Virus Growth Reduction Assay Protocol. Vero cells were plated in a 96-well plate at a concentration of 3×10⁵ cells/mL in 0.1 mL and incubated overnight at 37° C., 5% CO₂. Compounds to be tested were prepared in DMEM with 10% FBS. Each compound was tested at 10 μg/ml final concentration. One hundred μL of each compound dilution were added to the Vero cells and the cells were incubated for 45-60 minutes at 37° C., 5% CO₂. Vesicular Stomatitis Virus (Indiana strain) was added to the cells (125 PFU/well) and the plates were incubated at 37° C. for 24 hours. Supernatant containing the virus was removed from each well and a standard plaque assay was performed.

Standard Plaque Assay for VSV Using Vero Cells. Vero cells were seeded in a 6-well microtiter plate at a concentration of 2.5×10⁵ cells/mL in 2 mL/well (3×10⁶ cells/6-well plate). A 1:10 to 10⁻⁸ dilution series of the above-referenced supernatant containing virus was prepared in DMEM-2 (2% FBS 2% HEPES+1% Pen/Strep). Medium was removed from wells and replaced with 500 μL of the virus dilution series (in duplicate). Plates were incubated for 1 hour at 37° C. with gentle rocking every 10-15 minutes. Subsequently, approximately 3 mL of a 2% LMP agarose overlay (equal amounts of 2% agarose and 2× DMEM-COMPLETE [4% FBS+4% HEPES+2% Pen/Strep+9.8% Sodium Bicarbonate (of a 7.8% Solution)]) was added to each well. Once the agarose had solidified, the plates were incubated at 37° C. for 36 hours. The plates were stained by adding 2 mL of crystal violet solution and incubating at room temperature for about 4 hours. Plates were subsequently washed 2× with water plaques were counted.

Results. The results of this analysis demonstrate that the compounds herein exhibit antiviral activity (Table 9).

TABLE 9

| Compound* | VSV (PFU/mL) | % Plaque Reduction |
|---|---|---|
| 5 | 3.2 × 10E⁸ | 5 4.2 |
| 14 | 1.78 × 10E⁸ | 29.7 |
| 16 | 4.8 × 10E⁸ | 81.8 |
| 18 | 6.8 × 10E⁸ | 99.95 |
| 19 | 4.5 × 10E⁸ | 76.6 |
| 21 | 2.3 × 10E⁸ | 38.7 |
| Virus Control | 5.8 × 10E⁸ | none |

*Tested at 10 μg/mL final concentration.

what is claimed is:

1. A method of controlling dismutation in the synthesis of a heteroleptic triarylbismuthane comprising adding to a nucleophile a substoichiometric amount of a diarylbismuth or arylbismuth precursor relative to said nucleophile thereby controlling dismutation of the heteroleptic triarylbismuthane.

2. The method of claim 1, wherein the diarylbismuth or arylbismuth precursor has the structure of Formula I 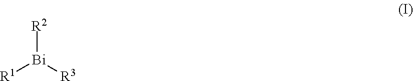
wherein R¹ is a substituted or unsubstituted aryl; R² is a leaving group; and R³ is the same as R¹ or R².

3. The method of claim 2, wherein R¹ is a substituted or unsubstituted aryl; R² is a tosyl group; and R³ is the same as R¹ or R².

4. The method of claim 1, wherein the nucleophile is an organometal.

5. The method of claim 4, wherein the organometal is an organozinc, organomagnesium or organocuprate.

6. The method of claim 1, wherein the heteroleptic triarylbismuthane has the structure of Formula II 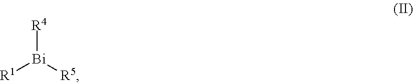
wherein R¹ and R⁴ are independently a substituted or unsubstituted aryl; R⁵ is the same as R¹ or R⁴; and R¹ and R⁴ are different.

7. A compound having the structure of Formula II 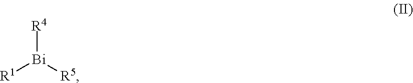
wherein R¹ and R⁴ are independently a substituted or unsubstituted aryl or substituted or unsubstituted arylsulfonate; and R⁵ is the same as R¹ or R⁴, wherein at least one of R¹ or R⁴ is

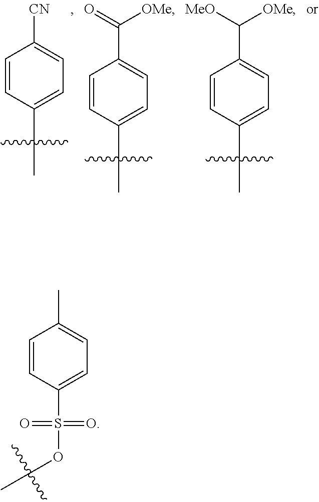

8. A pharmaceutical composition comprising a compound of claim 7 in admixture with a pharmaceutically acceptable carrier.

9. The pharmaceutical composition of claim 8, wherein said pharmaceutical composition is provided in the form of a tablet, capsule, pellet, lozenge or powder.

10. A method for inhibiting the replication of a microorganism comprising contacting a microorganism with a compound of claim 7, thereby inhibiting the replication of the microorganism.

11. The method of claim 10, wherein the microorganism is a virus or bacterium.
